SEQUENCE LISTING (15 sequences)

SEQ ID NO: 1 - protein, 141 aa, Homo sapiens
SELKCDISAEIQQRQEELDAMTQALQALQEQDSAEGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLR

SEQ ID NO: 2 - protein, 138 aa, Mus sp.
SHLHCDIGEEIQQWHEELGTMTQTLEEQGRTFDSAHAQMCSAIGQLDHARADIEKQIGARVRQVVDYVQAQERELLEAVNDRYQRDYQEIAGQLSCLEAVLQRIRTSGALVKRMKLYASDQEVLDMHSFLRKALCSLR

SEQ ID NO: 3 - protein, 132 aa, Homo sapiens
FQEHKNHSTVTVEEAKAEKETELSLQKEQLQLKIIEIEDEAEKWQKEKDRIKSFTTNEKAILEQNFRDLVRDLEKQKEEVRAALEQREQDAVDQVKVIMDALDERAKVLHEDKQTREQLHSISDSVLFLQEF

SEQ ID NO: 4 - protein, 126 aa, Homo sapiens
RDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIAS

SEQ ID NO: 5 - protein, 146 aa, Homo sapiens
GRHRDHQVAALSERYDKLKQNLESNLTNLIKRNTELETLLAKLIQTCQHVEVNASRQEAKLTEECDLLIEIIQQRRQIIGTKIKEGKVMRLRKLAQQIANCKQCIERSASLISQAEHSLKENDHARFLQTAKNITERVSMATASSQ

SEQ ID NO: 6 - protein, 144 aa, Homo sapiens
DHQVASLNDRFEKLKQTLEMNLTNLVKRNSELENQMAKLIQICQQVEVNTAMHEAKLMEECDELVEIIQQRKQMIAVKIKETKVMKLRKLAQQVANCRQCLERSTVLINQAEHILKENDQARFLQSAKNIAERVAMATASSQVL

SEQ ID NO: 7 - protein, 67 aa, Mus sp.
CQLNAHKDHQYQFLEDAVRNQRKLLASLVKRLGDKHATLQKNTKEVRSSIRQVSDVQKRVQVDVKMA

SEQ ID NO: 8 - protein, 164 aa, Homo sapiens
DLEATLRHKLTVMYSQINGASRALDDVRNRQQDVRMTANRKVEQLQQEYTEMKALLDASETTSTRKIKEEEKRVNSKFDTIYQILLKKKSEIQTLKEEIEQSLTKRDEFEFLEKASKLRGISTKPVYIPEVELNHKLIKGIHQSTIDLKNELKQCIGRLQELTP

SEQ ID NO: 9 - protein, 130 aa, Mus sp.
LSQASADLEYKLRNKLTIMHSHINGATKALEDVRSKQQCVQDSMKRKMEQLRQEYMEMKAVIDAAETSSLRRLKEEEKRVYGKFDTIYQVLVKKKSEMQKLKAEVELIMDKGDEFEFLEKAAKLQGESTK

SEQ ID NO: 10 - protein, 106 aa, Xenopus laevis
EASLKVTEQLSSEQSDKIEQHNKNMSQYKEHITSEFEKLHKFLREREEKLLEQLKEQGENLLTEMENNLVKMQESQDAIKKTISLAKERMEDTDSISFLMDIKAFI

SEQ ID NO: 11 - protein, 121 aa, Homo sapiens
FRINEVVKECQEKLQVALQRLIKEDQEAEKLEDDIRQERTAWKIERQKILKGFNEMRVILDNEEQRELQKLEEGEVNVLDNLAAATDQLVQQRQDASTLISDLQRRLTGSSVEMLQDVIDV

SEQ ID NO: 12 - protein, 136 aa, Mus sp.
LCERSQEHRGHQTALIEEVDQEYKEKLQGALWKLMKKAKICDEWQDDLQLQRVDWENQIQINVENVQRQFKGLRDLLDSKENEELQKLKKEKKEVMEKLEESENELEDQTELVRDLISDVEHHLELSTLEMLQGAN

SEQ ID NO: 13 - protein, 125 aa, Homo sapiens
LEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLE

SEQ ID NO: 14 - protein, 65 aa, Homo sapiens
ETWRRGDALSRLDTLETSKRKSLQLLTKDSDKVKEFFEKLQHTLDQKKNEILSDFETMKLAVMQA

SEQ ID NO: 15 - protein, 120 aa, Homo sapiens
EHREHGTVLLRDVVEQHKAALQRQLEAVRGRLPQLSAAIALVGGISQQLQERKAEALAQISAAFEDLEQALQQRKQALVSDLETICGAKQKVLQTQLDTLRQGQEHIGSSCSFAEQALRL

(19) United States
(12) Patent Application Publication
(10) Pub. No.: US 2003/0207791 A1
(43) Pub. Date: Nov. 6, 2003

(54) Materials and methods relating to the treatment of leukaemias

(76) Inventors: Saverio Minucci, Opera (IT); Pier Giuseppe Pelicci, Opera (IT)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/239,608

(22) PCT Filed: Mar. 22, 2001
(86) PCT No.: PCT/GB01/01274

(30) Foreign Application Priority Data
Mar. 24, 2000 (GB) 0007217.3

(51) Int. Cl. A61K 31/00; G01N 33/574

(52) U.S. Cl. 514/1; 435/7.23

(57) ABSTRACT

The invention provides materials and methods capable of modulating the strong-self-association of chimeric transcription factors to form high molecular weight (HMW) complexes. The invention further provides compounds comprising the oligomerization domains of oligomeric substances and a polypeptide for modulating the activity of that polypeptide intra or inter-cellularly.

### FIELD OF THE INVENTION

The present invention relates to the materials and methods involved in the treatment of leukaemias. Particularly, but not exclusively, the present invention relates to materials and methods capable of modulating the strong self-association of chimeric transcription factors to form high molecular weight (HMW) complexes as compared to the naturally occurring monomeric transcription factor. The present invention is primarily concerned with those transcription factors involved in differentiation of primary hematopoietic precursors.

### BACKGROUND OF THE INVENTION

Acute myeloid leukaemias (AMLs) are characterised by chromosomal translocations resulting in the generation of chimeric genes and fusion proteins (Look, 1997; Rabbitts, 1994; Rabbitts, 1991; Tenen et al., 1997). Ectopic expression of fusion proteins induces differentiation block of hemopoietic precursors and leukemias in animal models (Du et al., 1999; Gelmetti et al., 1998; Grignani et al., 1993; Grignani et al., 1996; Lavau et al., 1997; Pereira et al., 1998; Ruthardt et al., 1997; Schwaller et al., 1998; Slanyet al., 1998; Brown et al., 1997; Grisolano et al., 1997; Westervelt and Ley, 1999). One of the genes involved in the AML-associated translocations encodes almost invariably for a transcription factor, which is physiologically involved in hematopoietic differentiation (such as retinoic acid receptor α—RARAα—in acute promyelocytic leukaemia—APL—, or AML-1 in acute myelogenous leukaemia: Look, 1997; Rabbitts, 1994; Rabbitts, 1991; Shivdasani and Orkin, 1996; Tenen et al., 1997). According to the current model of leukaemogenesis, the differentiation block is the consequence of the altered transcriptional properties of these chimeric transcription factors (Look, 1997; Shivdasani and Orkin, 1996; Tenen et al., 1997). The molecular mechanisms of the oncogenic conversion, however, are largely unknown.

Recent findings demonstrated that aberrant recruitment of the nuclear corepressor (NCoR)-histone deacetylase (HDAC) complex is crucial to the activation of the leukemogenic potential of RAR and AML 1 in the fusion proteins PML-RAR and AML 1-ETO (Cheng et al., 1999; David et al., 1998; Gelmetti et al., 1998; Grignani et al., 1998; Guidez et al., 1998; He et al., 1998; Lutterbach et al., 1998; Wang et al., 1998). Histone acetylation levels influence chromatin structure in a manner tightly linked to transcriptional activity: high levels of histone acetylation are observed at the promoters of transcribed genes, whereas hypo-acetylation has been correlated to silenced genes (Grunstein, 1997; Pazin and Kadonaga, 1997). It is expected, therefore, that modification of the chromatin structure at the target promoters of the fusion proteins represents one important mechanism of leukaemogenesis (Minucci and Pelicci, 1999; Redner et al., 1999; Stunnenberg et al., 1999).

Unliganded RARs repress transcription by recruiting the NCoR/HDAC complex: RA triggers dissociation of the NCoR/HDAC complex and recruitment of several co-activators (PCAF, p300/CBP, SRC-1) endowed with histone acetylase activity , thus leading to transcriptional activation (Chambon, 1996; Mangelsdorf and Evans, 1995; Minucci and Pelicci, 1999; Wolffe et al., 1997; Xu et al., 1999). AML 1 is a transcriptional activator associated with p300/CBP (Kitabayashi et al., 1998), while ETO interacts with NCoR and recruits HDAC activity in vivo (Gelmetti et al., 1998; Lutterbach et al., 1998; Wang et al., 1998). The PML-RAR fusion protein retains the NCoR binding site of RAR, whereas AML 1-ETO has lost the p300/CBP interaction site of AML 1 but retains the NCoR/HDAC binding site of ETO (Minucci and Pelicci, 1999). In line with these findings, PML-RAR and AML 1/ETO form stable complexes with NCoR-HDAC (Gelmetti et al., 1998; Grignani et al., 1998; He et al., 1998; Lin et al., 1998; Lutterbach et al., 1998). Mutation of the NCoR binding site(s) impairs the biological activity of the two fusion proteins, indicating that formation of aberrant complexes with histone-modifying enzymes is essential for leukaemogenesis (Gelmetti et al., 1998; Grignani et al., 1998).

The mechanisms leading to abnormal recruitment of the NCoR/HDAC complex differ in the case of PML-RAR or AML 1-ETO. In AML 1-ETO, loss of p300/CBP and gain of NCoR association might be sufficient to endow the fusion protein with constitutive transcriptional repressive activity. In contrast, PML-RAR has the same property of RAR to recruit NCoR in the unliganded state: how the association with NCoR becomes abnormal when RAR is fused to PML remains unclear.

### SUMMARY OF THE INVENTION

The present inventors have for the first time established that the formation of HMW complexes of chimeric transcription factors (PML-RAR and AML1-ETO) results in abnormal recruitment of the NCoR/HDAC complex. This discovery has provided an important insight into mechanisms leading to the production of HMW complexes of chimeric transcription factors, e.g. PML-RAR and AML1-ETO, and as a result the abnormal recruitment of the NCoR-HDAC. This knowledge would have a number of important and industrially applicable implications, particularly as regards the treatment or diagnosis of leukaemias. Further, an understanding of the mechanisms involved in the formation of HMW complexes of oligomeric factors leading to their altered activity, opens the way to identifying a domain within other naturally occurring oligomeric factors e.g. chimeric transcription factors or other classes of proteins. This domain may then be used as a tool to enhance, through self-association, the functional properties of a given protein, not already present in nature as a strongly self-associating factor. In other words, manipulation of this domain may be equivalent (for the function of a protein) to genetically manipulating a promoter for a gene that normally (unmanipulated) has a weak promoter, to make it stronger. A further extension of the studies has also led the inventors to the conclusion that use of said domain to promote self-association, especially in the case of proteins already present as oligomeric complexes in nature, may lead through an oligomerization chain reaction to a reduced activity of a given protein.

The inventors have found that PML-RAR (unlike RAR) forms tightly interacting oligomers in vivo and that the coiled coil region of PML is the structural determinant for strong self-association and oligomerization. They have been able to show that oligomerization is responsible, per se, for (a) the increased recruitment of NCoR, (b) constitutive transcriptional repressive activity on RA-target promoters; and (c) leukemogenic potential of the fusion protein. A similar potential to form oligomeric structures has also been observed for the other APL-associated (PLZF-RAR and NPM-RAR) fusion proteins. AML1-ETO was also found in HMW complexes and shown to form oligomeric complexes, owing to the ETO moiety of the fusion protein. A derivative of AML 1-ETO devoided of the capacity to form HMW complexes showed a decreased capacity to interact with NCoR, impaired transcriptional repressive activity and was unable to block terminal differentiation of hematopoietic precursors. These findings highlight the physical status of a transcription factor as a potent mechanism to modulate its ability to recruit co-regulators, and indicate that self-association/oligomerization by heterologous interaction interfaces is a novel mechanism for the oncogenic conversion of a transcription factor in leukaemias. Thus, having for the first time determined this mechanism, the inventors have realised that disruption or inhibition of the formation of unwarranted oligomeric complexes provides a target for therapeutical intervention in the treatment of this disease. The information provided herein allows for the provision of materials and methods for (i) affecting the biological pathway involved in differentiation of primary hematopoietic precursors; (ii) affecting the biological pathway(s) involved in oncogenic transformation by altered transcription factors; (iii) assessing the presence of transcription factors with the above-mentioned altered properties in cancer samples; (iv) modifying the activity of a given protein by fusion with the heterologous coiled coil domain from PML with the intent of enhancing its functional activity or to reduce its functional activity; (v) modifying the activity of a given protein by fusion with the heterologous coiled coil domain from PML with the intent of reducing its functional activity.

Thus, in summary, a specific domain (termed hereinafter “oligomerization domain”) within transcription translocation proteins (e.g. PML-RARα), implicit in the cause of leukaemias represents the necessary means by which these proteins self associate with each other. This is known to occur prior to binding to the DNA where the fusion protein inhibits DNA transcription and thereby brings about the phenotypic changes manifested in leukaemic patients e.g. loss of differentiation. The inventors have shown for the first time that loss of this oligomerization domain and concomitant loss of the ability to form self-associating homodimers is sufficient to render these mutated proteins harmless and restore the cancerous cells back to their normal differentiated state. Until now it has never been shown that the formation of these oligomers with the enhanced capacity to recruit NcoR is a prerequisite for disease progression. Surprisingly, if the diseased translocation proteins remain as monomeric or single units they have no debilitating effects and normal cell differentiation occurs.

Therefore, at its most general, the present invention provides materials and methods which detect or affect the formation of tightly self-interacting oligomeric complexes. In particular, the invention is concerned with oligomeric complexes of chimeric factors, e.g. transcription factors.

A chimeric transcription factor is a fusion protein comprising a transcription factor or part thereof and a second protein—that may—or may not—be a transcription factor itself. The fusion protein is encoded by a gene altered as a result of a translocation event. These chimeric transcription factors have altered activity with respect to the wild type transcription factor. Examples of chimeric transcription factors include PML-RAR and AML1-ETO.

Preferably the chimeric transcription factors are products of the chromosomal translocations associated with leukaemias. Even more preferably the chimeric transcription factors are PML-RAR and AML1-ETO.

Oligomerization (trimers or hexamers in PML-RAR's case, but could be dimers with a different “n” oligomerization number) is critical to leukaemogenesis due to the increased concentration of binding sites for co-regulatory factors including NCoR which binds HDAC. HDAC has been shown to inhibit transcription. Thus, owing to the increased local concentration of NCoR and/or because of increased stability of NCoR binding (since as soon as one molecule disassociates there is another binding site very close by to which it can bind), the addition of RA at natural concentrations is no longer sufficient enough to replace the NCoR/HDAC and allow transcription to proceed. Moreover, oligomerization of PML-RAR and AML1-ETO transcription factors, through the avidity component (owing to multimerization of the NCoR binding sites) and through entropic effects (owing to an increase in the local concentration of NCoR binding sites), leads to a dramatic increase in the stability of their interaction with transcriptional co-repressors and possibly other co-regulators, thus leading to deregulated transcription.

This oligomerization principle is shown herein in the fusion proteins PML-RAR and AML1-ETO but is likely to be true in other leukaemia associated translocation proteins involving transcription factors and therefore in any number of diseases where translocation proteins are involved. For convenience, the text concentrates on chimeric transcription factors as the oligomeric factors. However, the skilled person will appreciate that the aspects of the invention may be applied to other oligomeric factors, e.g. TNF, p53 etc. In the context of this invention, an oligomeric factor is a polypeptide including a chimeric or fusion polypeptide that is capable of binding to other oligomeric factors to form an oligomeric complex. A monomeric factor is a polypeptide that exists as a single entity and does not naturally form complexes, e.g. thyroid receptor.

Thus, in a first aspect of the present invention there is provided a method of determining the presence or absence of a High Molecular Weight (HMW) complex comprising a chimeric transcription factor, preferably PML-RAR or AML 1-ETO, comprising the steps of obtaining a biological sample from a patient and detecting the presence or absence of said HMW complex. A HMW complex comprises two or more oligomeric factors, e.g. chimeric transcription factors which form a tightly self-interacting oligomeric complex. The HMW complex may comprise dimers, trimers, tetramers, pentamers, hexamers etc, of the oligomeric factors, e.g. chimeric transcription factor.

The complex may be detected using standard techniques known to those skilled in the art, such as using a specific binding member capable of binding to the complex, e.g. an antibody binding domain, the specific binding member being labelled so that binding of the specific binding member to the complex is detectable. For example, in the case of acute promyelocytic leukaemia (APL) chimeric transcription factors (PML-RAR, PLZF-RAR, NPM-RAR and NuMA-RAR), the specific binding member may be labelled (radioactively, fluorescently etc.) retinoic acid that binds the RAR moiety of the chimeric transcription factors.

It may also be necessary to further determine the molecular weight of the product specifically bound by the specific binding member. This will serve to distinguish between detection of the HMW complex of interest and naturally occurring chimeric transcription factors which have not formed HMW complexes by, for example oligomerization, or even wild type non-chimeric transcription factors. The molecular weight of the bound product may be determined by, for example, size-exclusion chromatography and subsequent analysis of the column fractions by retinoic acid labelling, immuno-based detection techniques-Western blot, or ELISA.

Other techniques exist which may be used to indicate the oligomeric state of the HMW complex detected in the biological sample. These include determining stokes radius, or sedimentation coefficient etc. Other techniques will be apparent to the skilled person.

In order to determine the presence of absence of the HMW complexes in a biological sample, a comparison may be made with a control sample of known molecular weight of the chimeric transcription factors which have not formed HMW complexes and the wild type non-chimeric transcription factors.

The choice of biological sample will depend on the HMW complex being determined. For example, if the complex is formed by chimeric transcription factors PML-RAR or AML 1-ETO then the biological sample would preferably be blood. However, other examples of biological fluids include plasma, serum, tissue sample, tumour samples, saliva and urine.

This aspect of the invention may be used to diagnose a patient suspected of having an abnormality in the transcriptional control of certain genes due to abnormal chromosomal translocations, leading to the development of a disease such as cancer, or it may be used to determine the susceptibility of a patient to a particular form of disease e.g. cancer. For example, one could determine the presence or absence of HMW transcription factor complexes, reflecting their oligomeric nature. This determination has the advantage of being able to not only confirm the abnormality but also to determine the exact type of abnormality and, as a consequence, direct the specific treatment of the patient. For example, if a patient is suspected of having a form of acute leukaemia, a blood sample may be obtained and tested for the presence or absence of HMW chimeric transcription factor complexes (e.g. comprising PML-RAR or AML 1-ETO). Should any abnormal HMW chimeric transcription factor complexes be found then the patient may, following additional cytogenetical analysis if necessary, be diagnosed as having, or susceptible for, acute myeloid leukaemias (AML) or acute promyelocytic leukaemia (APL). In addition, the screening for the presence of HMW chimeric transcription factor complexes might be extended to other forms of cancer, where the detection of such complexes could also represent a critical factor for the therapeutical strategy.

Thus, an embodiment of this aspect of the present invention provides a method of diagnosing an acute myeloid leukaemia or APL comprising the steps of obtaining a biological sample from a patient, preferably blood or serum, and testing said sample for the presence of a HMW complex comprising PML-RAR or AML 1-ETO.

In a second aspect of the present invention there is provided a method of treating a patient having a disease, such as cancer, associated with the formation of HMW complexes comprising chimeric transcription factors thereby resulting in the abnormal transcriptional control of gene(s), said method comprising administering to said patient a factor capable of disrupting the activity or formation of said HMW complexes. Preferably, the factor prevents, disrupts or inhibits the formation of the HMW complex. For example, if the complex was formed through the oligomerization of PML-RAR then the factor may be capable of preventing or disrupting the oligomerization. The present inventors have discovered for the first time that in the case of PML-RAR the structural determinant of oligomerization of the chimeric transcription factor, as well as of the natural PML protein, is the coiled coil region of PML. Thus, an embodiment of this aspect of the present invention would be to block the activity of this region of the factor in question, e.g. PML, such that oligomerization could not take place.

This disruption is preferably achieved by the administration of factors such as binding members which are capable of specifically binding to the coiled coil region of PML such that oligomerization cannot take place. Examples of such binding members include (I) antibody binding domains specific for an epitope in the region in question; (ii) oligopeptides comprising the coiled coil domain of PML itself in the case of PML-RAR (and therefore capable of binding PML-RAR HMW complexes and disrupting them), or the self association domain specific for other chimeric transcription factor; (iii) small molecules derived from screening for compounds exhibiting the capability of preventing/disrupting specific HMW complexes (see below).

In this context, disruption may be taken to mean either the prevention of complex formation or, if the complex has already formed, the prevention of complex activity such as transcriptional repressive activity. Prevention of complex activity may be achieved by break-up of the complex itself.

As mentioned above, the present inventors have determined for the first time that the formation of these HMW complexes (including oligomerization) are responsible for (a) the increased recruitment of NCoR; (b) the localised increase in HDAC concentration; (c) the constitutive transcriptional repressive activity; and (d) the leukemogenic potential of the fusion protein of the chimeric transcription factors. Thus, the inventors have determined that as a result of the HMW complex formation, these fusion proteins have an increased capacity to interact with NCoR.

As also mentioned above, the present inventors have shown that for the PML-RAR fusion protein the structural determinant of oligomerization (oligomerization domain) of the chimeric transcription factor is the coiled coil region of PML, and that the oligomerization domain of AML1-ETO comprehends a coiled coil region. In the case of the other APL fusion proteins, NuMA-RAR, PLZF-RAR, and NPM-RAR, the oligomerization domain contributed by NuMA is a coiled coil region, whereas PLZF and NPM show a different folding of their oligomerization domains. The oligomerization domain (or coiled coil) is the structural determinant for strong self-association and oligomerization.

In PML this coiled coil domain has the following amino acid sequence (SEQ ID NO 1).

SELKCDISAEIQQRQEELDAMTQALQALQEQDSAEGAVHAQMHAAVGQLG
RARAETEELIRERVRQVVAHVRAQERELLEAVDARYQRDYEEMASRLGRL
DAVLQRIRTGSALVQRMKCYASDQEVLDMHGFLRQALCRLR

The murine coiled coil domain of PML has the following amino acid sequence (SEQ ID NO 2).

SHLHCDIGEEIQQWHEELGTMTQTLEEQGRTFDSAHAQMCSAIGQLDHAR
ADIEKQIGARVRQVVDYVQAQERELLEAVNDRYQRDYQEIAGQLSCLEAV
LQRIRTSGALVKRMKLYASDQEVLDMHSFLRKALCSLR

Thus, the present invention further provides assays using a peptide (produced in vitro, or in vivo through methods available to the skilled person) having either the murine or human sequence given above, or a variant thereof to find substances capable of modulating the oligomerization domain so that self-association of the chimeric transcription factors is prevented or reduced. Analogously, the present invention further provides assays using a peptide having sequences corresponding to the oligomerization domain of any given chimeric transcription factor, or a variant thereof, to find substances capable of modulating the oligomerization domain so that self-association of the chimeric transcription factors is prevented or reduced.

One class of substance that may be used to disrupt the oligomerization domain are peptides based on the sequence motifs of the coiled coil region which causes oligomerization/strong self-association. Such peptides tend to be small molecules, and may be about 40 amino acids in length or less, preferably 35 amino acids in length more preferably 30 amino acids in length, or less, more preferably 25 amino acids in length or less, more preferably 20 amino acids in length or less, more preferably about 15 amino acids or less, more preferably about 10 amino acids or less, or 9, 8, 7, 6 5 or less in length. The present invention also encompasses peptides which are sequence variants or derivatives of a wild type oligomerization domain, i.e. the coiled coil domain as given above.

Preferably, the amino acid sequence shares homology with a fragment of the coiled coil domain sequence shown preferably at least about 30%, or 40%, or 50%, or 60%, or 70%, or 75%, or 80%, or 85% homology, or at least about 90% or 95% homology. Thus, the coil coiled domain of the chimeric transcription factor may include 1, 2, 3, 4, 5, greater than 5, or greater than 10 amino acid alterations such as substitutions with respect to the wild-type sequence.

As is well-understood, homology at the amino acid level is generally in terms of amino acid similarity or identity. Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Similarity may be as defined and determined by the TBLASTN program, of Altschul et al, J. Mol. Biol., 215:403-10, 1990, which is in standard use in the art. Homology may be over the full-length of the relevant peptide or over a contiguous sequence of about 5, 10, 15, 20, 25, 30 or 35 amino acids, compared with the relevant wild-type amino acid sequence.

Further, other small molecules or compounds may be used to modulate the structural determinant of oligomerization such that self-association of the chimeric transcription factors cannot take place. For example, lipids, phospholipids, oligosaccarides etc may be used. These molecules may have the advantage of possibly being easier to produce and deliver than peptides.

In one general aspect, the present invention further provides an assay method for a substance with ability to modulate the structural determinant of oligomerization (oligomerization domain) of an oligomeric factor such that strong self-association of the oligomeric factors to form oligomeric complexes is prevented or reduced, the method including:

(a) bringing into contact a first oligomeric factor or the functional self-association part thereof, a second oligomeric factor the functional self-association part thereof, and a test compound, under conditions wherein, in the absence of the test compound being an inhibitor of association of said oligomeric factors, said oligomeric factors or functional self-association parts thereof interact or bind; and,

(b) determining interaction or binding between said oligomeric factors or functional self-association parts thereof.

It will be apparent to the skilled person that to perform an assay method as defined above, the whole oligomeric factor need not be used. Indeed, it would be sufficient to use that part of the factor that is involved with the oligomerization/self-association of that factor. Thus, in the case of PML it would be possible to use a peptide comprising the coiled coil region of this transcription factor or even a fragment of this region known to be involved in oligomerization/self association. Any assay developed with an isolated region known to be involved in oligomerization/self-association will be subsequently extended to the whole transcription factor.

A test compound which disrupts, reduces, interferes with or wholly or partially abolishes binding or interaction between said monomeric chimeric transcription factors, and which may modulate the bioactivity of said transcription factors, may thus be identified.

Another general aspect of the present invention provides an assay method for a test compound able to bind the relevant region of the oligomerization domain (e.g. the coiled coil domain); the method including:

(a) bringing into contact a substance which includes a oligomerization domain(e.g. the coiled coil domain) which allows self-association of the oligomeric factors e.g. chimeric transcription factors, or a variant, derivative or analogue thereof, and a test compound; and,

(b) determining binding between said oligomerization domain and the test compound.

A test compound found to bind to the relevant portion of the oligomerization domain may be tested for ability to disrupt self-association of the oligomeric factors under test and/or the ability to affect the bioactivity or other activity mediated by the transcription factors.

Performance of an assay method according to the present invention may be followed by isolation and/or manufacture and/or use of a compound, substance or molecule which tests positive for ability to interfere with the self-association of the oligomeric factors and/or modulate their bioactivity.

The precise format of an assay of the invention may be varied by those of skill in the art using routine skill and knowledge. For example, interaction between substances may be studied in vitro by labelling one with a detectable label and bringing it into contact with the other which has been immobilised on a solid support. Suitable detectable labels, especially for petidyl substances include ³⁵S-methionine which may be incorporated into recombinantly produced peptides and polypeptides. Recombinantly produced peptides and polypeptides may also be expressed as a fusion protein containing an epitope which can be labelled with an antibody.

The protein which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the material attached to the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Alternatively, FRET (Fluorescence Resonance Energy Transfer)—based assay may be developed that will show when and how strongly these coiled-coil domains are self associating in vitro i.e. on easy to handle and high-throughput microwell plates.

An assay according to the present invention may also take the form of an in vitro assay. The in vivo assay may be performed in a cell line such as a yeast strain or mammalian cell line in which the relevant polypeptides or peptides are expressed from one or more vectors introduced into the cell. In this case, the demonstration of the interaction—or its prevention, or its disruption by the screened compounds—between the self-associating moieties under study will have the form of measurable enzymatic activity from a reporter enzyme, or fluorescence, or FRET phenomena.

In a third aspect of the present invention there is provided an assay method for a substance with the ability to disrupt interaction or binding between NCoR and a HMW complex formed from a chimeric transcription factor, for example, PML-RAR or AML 1-ETO, the method including

(a) bringing into contact said HMW complex or a variant, derivative, or analogue thereof, including the binding region for NCoR, a substance including the relevant fragment of NCoR or a variant, derivative or analogue thereof, and a test compound, under conditions wherein, in the absence of the test compound being an inhibitor of interaction between or binding of said substances, said substances bind; and

(b) determining the interaction or binding between said substances.

A test compound which disrupts, reduces, interferes with or wholly or partially abolishes binding or interaction between said substances (e.g. including a NCoR binding site on a HMW complex and NCoR) and which modulate the transcriptional repressive activity resulting from such interaction, may be identified.

Again, performance of an assay method according to the present invention may be followed by isolation and/or manufacture and/or use of a compound, substance or molecule which tests positive for ability to interfere with interaction between the HMW complex and NCoR and/or inhibit biological activity, i.e. transcriptional repressive activity.

An assay according to the present invention may also take the form of an in vivo assay. The in vivo assay may be performed in a cell line such as a yeast strain or mammalian cell line in which the relevant polypeptides or peptides are expressed from one or more vectors introduced into the cell. In this case, the demonstration of the interaction—or its prevention, or its disruption by the screened compounds—between the factors under study will have the form of measurable enzymatic activity from a reporter enzyme, or fluorescence, or FRET phenoma.

Antibodies directed to the site of interaction in either the HMW complex or NCoR form a further class of putative inhibitor compounds. Candidate inhibitor antibodies may be characterised and their binding regions determined to provide single chain antibodies and fragments thereof which are responsible for disrupting the interaction.

Antibodies generated during all of the previously described assays may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature 357:80-82, 1992). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Following identification of a substance or agent which modulates or affects the transcriptional repressive activity. e.g. the disruption of the coiled coil region or similar oligomerization domains, the substance or agent may be investigated further. Derivatives with higher activity, or improved pharmaco-kinetic properties, may be obtained; Furthermore, it may be manufactured and/or used in preparation, i.e. manufacture or formulation, of a composition such as a medicament, pharmaceutical composition or drug. These may be administered to individuals.

Generally, a substance or agent which is capable of inhibiting, modulating or affecting the transcriptional repressive activity of the HMW complexes according to the present invention may be provided in an isolated and/or purified form, i.e. substantially pure. This may include being in a composition where it represents at least about 90% active ingredient, more preferably at least about 95%, more preferably at least about 98%. Such a composition may, however, include inert carrier materials or other pharmaceutically and physiologically acceptable excipients. As noted below, a composition according to the present invention may include in addition to an inhibitor compound as disclosed, one or more other molecules of therapeutic use, such as an anti-cancer agent.

The present inventors have further determined that fusion of the coiled coil region of PML to the human thyroid receptor (TR) results in a chimeric transcription factor with enhanced recruitment of NCoR, and enhanced transcriptional repressive properties (FIG. 8). It therefore follows that oligomerization through the strong self-associating coiled coil domain of molecules such as PML may enhance the functional activity of any given protein found in nature in a monomeric state, or in an unstable di- or multimeric state. The inventors have additionally considered that the oligomerization of monomeric factors (or factors in an unstable di- or multimeric state) will result in the formation of an oligomeric complex with enhanced activity only in case of the proper structural organization of the oligomer itself, and of its active interfaces: for example, it must be assured (through the addition of appropriate “hinge” regions) that the oligomerized interaction surfaces for associated factors will be correctly and spatially oriented.

Thus, in a fourth aspect of the present invention, there is provided a method of modifying the activity of a polypeptide by contacting said polypeptide under suitable conditions with a compound comprising an oligomerization domain such as the coiled coil region of PML. Preferably, the polypeptide is fused under suitable conditions with the coiled coil region of PML or variants/derivatives thereof, with the intent of enhancing the biological activity of said polypeptide.

However, the present inventors have also determined that the oligomerization domain of p53 fused to RAR may substitute for the coiled coil region of PML and as a result, shows enhanced recruitment of NCoR, enhanced transcriptional repressive properties and the block of hematopoitetic differentiation. Thus, the present inventors have surprisingly found that, although the nature of the oligomerization domain might differ from the coiled coil region of PML, the functional properties (for example, increased activity) remain the same.

By way of example, the present inventors have determined that the following oligomerization domains, when fused to RAR, result in a chimeric protein with increased activity and biological properties: a) the coiled coil region of PML; b) the oligomerization domain of NPM-nucleophosmim; c) the POZ domain of PLZF; the oligomerization domain of NuMA; d) the tetramerization domain of p53.

The oligomerization domains present in the above mentioned proteins are also found (with variable degrees of homology) in other proteins. For example, several proteins, including PML, are known which show the so-called tri-partite region, that includes a RING domain, a B-box(es) region, and the coiled coil (Saurin et al., 1996). Examples of these proteins, and of the corresponding coiled coil sequences are given in FIG. 9.

The inventors believe that the coiled coil regions in proteins which correspond to PML, examples of which are given in FIG. 9, mediate a similar function to the coiled coil region in PML. Therefore, in accordance with the present invention, the coiled coil regions of other proteins (in addition to PML) may be fused to target polypeptides or proteins to enhance their functional activity.

Thus, the fourth aspect of the present invention may be expanded to include a compound comprising a coiled coil region of a protein, said coiled coil region corresponding to that of the coiled coil region of PML. The protein may be one as exemplified in FIG. 9 or it may have, with regard to the coiled coil, structural and sequence similarity with the coiled coil region of PML. Preferably, the sequence homology with be at least 50% homology in amino acid sequence with the coiled coil region of PML. Preferably, the sequence homology will be at least 60%, more preferably at least 70% and even more preferably at least 80% or 90%.

As mentioned above, the inventors have show that fusion of the coiled coil domain of PML to RAR or TR increases the functional activity of these polypeptides. However, it will be appreciated by the skilled person that the applicability of these coiled coil regions need not be limited to these particular polypeptides. Indeed, the coiled coil regions may be used to increase the functional activity of other polypeptides/ proteins such as: a) molecules endowed with enzymatic activity, e.g. cre-recombinase, histone dacetylase; b) extracellular ligands for cell membrane receptors; c) other transcription factors (nuclear receptors, HOX genes); and d) therapeutic antibodies. The functional activity of such an antibody or part thereof (e.g. binding domain) will be increased owing to multiples of the antibody combining to form a single compound.

The fusion of the coiled coil region to the polypeptide in question may be achieved using standard molecular techniques known to the skilled person. For example, a plasmid may be generated to express the chimeric polypeptide/protein in bacteria or mammalian cells, where the coiled coil region or part thereof of, for example, PML is fused by standard molecular biological techniques to the desired polypeptides/proteins. Upon fusion with the coiled coil region of, for example PML, the chimeric protein may undergo in vitro and in vivo analysis for their biochemical and functional properties. For example, the chimeric protein may undergo any one or more of the following:

1) a demonstration of the oligomeric state of the chimeric protein compared with the natural protein;

2) functional tests such as a) in the case of enzymatic activities, enzymatic assays already available for the natural proteins; b) in the case of extracellular ligands, e.g. cytokines, the activation of the corresponding receptor, and the biological consequences of the activation—e.g. apoptosis, differentiation, or other phenomena; c) in the case of transcription factors, recruitment of coregulators (NCoR, HDACs, CBP/p300, P/CAF), and measurement in transfection assays of transcriptional activity; d) in the case of therapeutic antibodies (or portions thereof), ELISA screening, neutralisation experiments or biological assays.

Therefore, the present invention further provides a method of enhancing the functional activity of a polypeptide, said method comprising producing a chimeric protein comprising a strong self-association domain (oligomerization domain) of a protein and said polypeptide, said chimeric protein not being present in nature as a multimeric complex. The strong self-association domain may be the coiled coil domain of PML or it may comprise other domains such as exemplified in FIG. 9 which are related to the coiled coil domain of PML, or domains consisting of a different primary sequence and structure, but exerting a similar function of induced oligomerization.

The present invention further extends in various aspects not only to a substance identified as a modulator of HMW complex formation and stability, or HMW complex/NCoR interaction or HMW complex/NCoR-mediated activity, property or pathway in accordance with what is disclosed. herein, but also a pharmaceutical composition, medicament, drug or other composition comprising such a substance, a method comprising administration of such a composition to a patient, e.g. for anti-cancer such as leukaemia, use of such a substance in manufacture of a composition for administration, e.g. for anti-leukaemia or similar treatment, and a method of making a pharmaceutical composition comprising admixing such a substance with a pharmaceutically acceptable excipient, vehicle or carrier, and optionally other ingredients.

The invention further provides a method of modulating the activity of HMW complexes which bind recruit and interact with NCoR, or other HMW complex mediated activity in a cell, which includes administering an agent which inhibits or blocks the binding of HMW complex such as PML-RAR and AML 1-ETO to NCoR, such a method being useful in treatment of leukaemias or other diseases or disorders including malignancies where transcriptional repressive activity is implicated.

The invention further provides a method of treating leukaemias which includes administering to a patient an agent which interferes with the binding of NCoR to HMW complexes comprising chimeric transcription factors such as PML-RAR and AML 1-ETO.

The present inventors have further determined that the addition of the coiled coil domain of PML to a “target” protein may result in functional inactivation of the target. They have shown that in the case of proteins oligomeric in nature (such as wild type p53), addition of an extra oligomerization domain (the coiled coil of PML) results in an oligomerization chain reaction not compatible with normal p53 localization and function. Thus, the inventors have determined that addition of an extra-oligomerization interface (a coiled-coil in accordance with the present invention) leads to the formation (through this oligomerization chain reaction) of high-order oligomeric. complexes, that results in the formation of non-functional aggregates. Put simply, the coiled-coil initiates a complexing of other oligomeric factors in its surrounding area which can serve to “mop up” (inactivate) unwanted protein in, e.g. a cell. The inventors have termed this technology “RITA” for Reaching (protein) Inactivation Through Aggregation. The RITA technology may therefore be applied to inactivate natural oligomeric proteins or any other protein that will be impaired functionally through this approach. Since the inventors have observed that the CC domain may mediate oligomerization also in the extracellular environment, this technique may be applied to both intra- and extra-cellular target proteins.

Thus, in a fifth aspect of the present invention, there is provided a method reducing the activity of a target oligomeric protein in an environment comprising the step of introducing a modified oligomeric protein into the environment, said modified oligomeric protein being the same protein as the target, or a functional fragment thereof, but comprising an additional oligomerization domain (coiled coil domain).

The environment may be a sample comprising a population of oligomeric proteins, e.g. an intracellular or extracellular environment. By addition of the modified oligomeric polypeptide to the sample, those oligomeric polypeptides present in the sample increase their oligomerization state to undesired levels and as a consequence their activity within the sample is decreased. The newly derived modified oligomeric protein may not comprise the whole of the protein in question, as long as its natural oligomerization domain, and eventual other domains (for example, a nuclear localisation signal) required for putting in contact said modified oligomeric protein with its natural counterpart, are maintained. The environment may be intra-cellular or extracellular. If the environment is intra-cellular, the modified oligomeric protein may be introduced into the cell by transfecting the cell with a vector comprising nucleic acid encoding it and the oligomerization domain. For example, if the target protein was wild type (wt) p53, then it may be desirable to introduce a fusion protein comprising the oligomerization domain and p53 (e.g. CC-p53). Such a fusion protein, or nucleic acid encoding the fusion protein may form a medicament for use in reducing the activity or inactivating oligomeric proteins in an environment e.g. a cell. Such medicaments may be used in the treatment of patients or they may be used for research purposes. For example, the ability to “mop up” unwanted protein in a cell provides an alternative to generating protein specific knock-out phenotypes. This may prove a faster and more practical preliminary test on the phenotypic importance of a new gene. This is a far simpler and faster alternative to generating knock out mice at the gene level, so often used now in functional genomics studies. In addition, it has the potential to be applied to human primary cells, or cell lines, and any other cell derived from a species for which the knock out technology is not available, or it is not ethically achievable (primates). Further, they have considerable application in the field of pharmacogenomics. The inventors have concentrated their studies on p53. However, the person skilled in the art will appreciated that many oligomeric factors exist and that therefore, this aspect of the invention may be applied to many factors e.g. cytokines, TNF, interleukins etc. The invention according to the fifth aspect may be used to “mop up” the wild type target protein and inactivate it in large multimeric complexes. This application would be particularly useful for inactivating systemic proteins involved in the immune system e.g. TNF and interleukins. This sort of application may be applied on a regulated and temporary basis to control graft or organ transplantation rejection or to treat autoimmune disorders such as rheumatoid arthritis.

Thus, the present invention further provides an oligomerization complex comprising an oligomeric factor and an oligomerization domain. The oligomeric factor is preferably selected from the group consisting of p53, interleukins, cytokines, TNF, etc. The oligomerization domain is preferably the structural determinant for strong self-association and oligomerization of the oligomeric factors e.g. the coiled-coil domain.

The present invention further provides a nucleic acid molecule (DNA, cDNA; RNA, or mRNA) which encodes an oligomerization complex as described above. The nucleic acid molecule may form part of an expression vector and may be operably linked to a promoter which can direct the expression of the nucleic acid. Thus, the present invention also provides a replicable vector comprising sequence encoding an oligomerization complex. The invention further provides a host cell transformed with the vector described above.

Whether it is a polypeptide, antibody, peptide, nucleic acid molecule (including vector/plasmid), small molecule, mimetic or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Targeting therapies may be used to deliver the active agent more specifically to certain types of cell, by the use of targeting systems such as antibody, cell specific ligands or viral vectors (in the case of polypeptides). Targeting may be desirable for a variety of reasons, for example if the agent is unacceptably toxic, or if it would otherwise require too high a dosage, or if it would not otherwise be able to enter the target cells.

### BRIEF DESCRIPTION OF THE DRAWINGS

Aspects and embodiments of the present invention will now be illustrated, by way of example, with reference to the accompanying figures. Further aspects and embodiments will be apparent to those skilled in the art. All documents mentioned in this text are incorporated herein by reference.

FIG. 1. Enhanced recruitment of NCoR by PML-RAR is due to the coiled coil region of PML. Increasing amounts of GST -NCoR (from 150 ng to 10 μg) coupled to agarose beads were incubated with the indicated in vitro translated, ³⁵-S labelled proteins. On the left, a schematic representation of the in vitro translated products, with the tripartite motif of PML highlighted (R, RING finger; B, B boxes; CC, coiled coil). The input lanes are loaded with the same amounts used in the pull-down experiments.

FIG. 2. Role of the coiled coil of PML in the altered transcriptional properties of PML-RAR (A): Transcriptional repression by RAR and fusion proteins. Hela cells were co-transfected with the RARE-G5-TATA reporter in the absence (C) or presence of increasing amounts (50, 100, 250, 1000 ng) of the indicated expression vectors and then harvested 48 hours after transfection. Levels of expression of RAR and fusion proteins after transfection are shown in (C). (B) RARE and NCoR dependent transcriptional repression by RAR and fusion proteins. Hela cells were co-transfected with the RARE-G5-TATA or the G5-TATA, reporters and 1 μg of control expression vector (C), RAR (R), PML-RAR (P/R) and their respective AHT derivatives, as indicated. (D) RA sensitivity of RAR and fusion proteins. Cells were co-transfected with 500 ng of the indicated expression vectors. 24 hours after transfection, RA was added at the following concentrations: 1, 10, 100 nM, 1, 10 μM, and cells were harvested after additional 24 hours.

FIG. 3. PML-RAR forms oligomeric complexes In vivo, which depend on the coiled coil of PML. (A) Role of the coiled coil of PML in the formation of HMW complexes. Nuclear extracts from NB4 cells or U937 clones expressing the indicated proteins (P/R, PML-RAR; CC/R, CC-RAR; ΔCC/R, ΔCC-PML-RAR) were fractionated by gel filtration chromatography. Fractions were analysed by Western blotting using an anti-RAR antibody. Fraction number is indicated at the top of each lane: Elution fractions of known molecular weight markers are indicated by arrows. (B) Recombinant PML-RAR forms oligomers. Fractions from gel filtration of in vitro translated, ³⁵-S labelled (ivt), or highly purified, bacterially expressed (BL21) PMl-RAR were analysed by SDS-PAGE, followed by autoradiography (ivt) or Western blotting using an anti-RAR antibody (BL21). (C) Biochemical purification of PML-RAR from U937 PR9 cells. A schematic diagram of the purification scheme is presented, together with the silver stain and the corresponding Western blot analysis of highly purified PMl-RAR after DNA-affinity chromatography. The bands marked by an asterisk are non-specific bands present also in the control sample. The material eluted from the DNA affinity chromatography was also analysed by gel filtration, followed by Western blot analysis. (D) In vivo cross-linking. Nuclear extracts from in vivo cross-linked, metabolically labelled U937 PR9 cells were analysed according to the flow-chart. Nuclear extracts were analysed by Western blot after SDS PAGE in reducing or non-reducing conditions: the arrows indicate the cross-linked species. Extracts were subjected to gel filtration chromatography and then analysed by Western blot after SDS PAGE in reducing or not reducing conditions. The pool of HMW PML-RAR complexes was immunoprecipitated with anti-PML or control (ctrl) antibodies and analysed by autoradiography following SDS PAGE in reducing conditions. An aliquot of the anti-PML immunoprecipitate was analysed by gel filtration, followed by SDS PAGE in reducing conditions and autoradiography. (E) Characterization of the oligomerization properties of the isolated coiled coil domain of PML. (Upper panel) Fractions from gel filtration of purified, bacterially expressed (BL21) CC domain of PML were analysed by SDS PAGE, followed by Western blotting using an anti-CC antibody. Purified CC was cross-linked in vitro with increasing concentrations of BS³. The silver stain (lower left panel) and the corresponding Western blot analysis (lower right panel) are shown. The position of the mono-, di- and tri-meric CC are indicated by arrowheads.

FIG. 4. PML-RAR oligomers associate with NCoR and DNA responsive elements. (A) In vivo association of HMW PML-RAR complexes with NCoR. Samples from metabolically labelled U937 PR9 cells (I, input lane) were immunoprecipitated with anti-NCoR or control (PI) antibodies (note: the PI lane derives from approximately 5 times more material than the specifically immunoprecipitated complexes). After extensive washing, the anti-NCoR containing beads were incubated in washing buffer in the presence of RA (10 μM) for 2 hours at 4° C. (RA). For comparison, the immunoprecipitate from the same cells using anti-PML antibodies is shown in the last lane (upper panel). The RA-eluted material was then analysed by gel filtration, followed by SDS PAGE and autoradiography (lower panel). (B) HMW PML-RAR complexes bind DNA. Mobility shift assays using the RARE from the RARβ2 promoter as a probe and extracts from Xenopus oocytes programmed with mRNA for RAR, PML-RAR either singly or co-injected with mRNA for RXR. Lanes 3-4, 5-8, 9-11 correspond to 0.03 (lane 5), 0.05 (lanes 6, 9),0.2 (lanes 3, 7, 10) and 0.5 (lanes 4, 8, 11) oocyte equivalent extract amounts. Arrows indicate the heterodimeric RXR-RAR and PML-RAR/RXR complexes, and the HMW PML-RAR and PML-RAR/RXR complexes. (C) HMW PML-RAR complexes recruit NCoR on DNA. Mobility shift assays on agarose gels using the RARE as a probe and extracts from Xenopus oocytes coinjected with mRNA for PML-RAR and RXR. Extracts were incubated with the labeled RARE in the presence of recombinant GST-NCoR (aa 1782-2453) or GST as a control. Where indicated, RA (10 μM) was added during the incubation. (D) HMW PML-RAR complexes do not contain NCoR or RXR. Western blot analysis using anti-NCoR (NCoR), anti-RAR (PML-RAR AHT) or anti-RXR antibodies (RXR) of fractions obtained from gel filtration analysis of nuclear extracts from U937 cells (NCoR and RXR) or from PML-RAR AHT expressing U937 cells. An identical elution profile of NCoR and RXR was obtained from PML-RAR expressing cells (data not shown).

FIG. 5. A heterologous oligomerization domain activates the transforming potential of RAR. (A-B) p53-RAR form oligomers. (A) Nuclear extracts from COS-1 cells transfected with expression vectors for p53-RAR or RAR were incubated with tritiated RA (10 nM), fractionated and analysed as described in FIG. 3A. Note that the low MW peak of RA-binding capacity (coinciding with RAR) in p53-RAR cells derives from expression of wild type RAR, starting from its own ATG-conserved in the p53-RAR expression vector (see below)-. (B) Fractions from gel filtration of in vitro translated, ³⁵-S labelled p53-RAR were analysed by SDS-PAGE, followed by autoradiography. Note that two products are generated in the reaction: full-length p53-RAR, fractionating in oligomeric complexes, and RAR, starting from the internal ATG site of RAR and fractionating as a monomer. (C-D) Effects of RAR chimeric proteins on differentiation of murine hematopoietic progenitors. Lin-cells were transduced with the indicated retroviral vectors and then sorted by FACS on the basis of their GFP positivity. In the case of ΔCC-PML-RAR, GFP+ cells were sorted in GFP^{high} or GFP^{low} expressors. After sorting, cells were either plated in differentiation medium in the absence or in the presence of RA (3 nM or 1 μM) (C), or analysed by Western blot (D). C, control, P-R, PML-RAR, ΔCC-P-R, ΔCC-PML-RAR, G-PR, GFP-PML-RAR, G-ΔCCPR, GFP-ΔCC-PML-RAR, G-p53R, GFP-p53-RAR. As described, RA delays myeloid differentiation of control cells, leading to a maximum 20-30% reduction in the number of Mac1 + cells (Purton et al., 1999). This effect is counter-acted by expression of PML-RAR and by high levels of ΔCC-PML-RAR. As described for wild-type RAR, expression of high levels of ΔCC-PML-RAR in the presence of physiological concentrations of RA relieves the differentiation block observed in the absence of ligand (Du et al., 1999).

FIG. 6. APL fusion proteins form HMW complexes due to the partners of RAR in the chromosomal translocations. (A) Nuclear extracts from COS-1 cells transfected with the indicated expression vectors were I) incubated with tritiated RA, fractionated and analysed as described in the methods section or ii) fractionated without prior incubation with RA, and analysed by Western blotting using anti-RAR antibodies. (B) Fractions from gel filtration analysis of nuclear extracts from COS-1 cells (NPM), or COS-1 cells transfected with PML or PLZF expression vectors were analysed by SDS-PAGE, followed by Western blotting. (C) PML is recruited to PML-RAR HMW complexes. Nuclear extracts from COS-1 cells co-transfected with expression vectors for PML and PML-RAR were fractionated by gel filtration and then analysed by SDS-PAGE, followed by Western blotting with anti-PML (that do not cross-react with the fusion protein data not shown) or anti-RAR antibodies. Alternatively, extracts were co-immunoprecipitated with anti-RAR antibodies, and the immunoprecipitated complexes were analysed by SDS-PAGE/Western, using anti-PML antibodies (that recognize both PML and PML-RAR proteins, Flenghi et al., 1993).

FIG. 7. Oligomerization of AML 1-ETO. (A) A schematic representation of AML 1-ETO and the deletions used: ZF, zinc fingers (NCoR interaction domain). (B) AML 1-ETO forms HMW complexes. AML1-ETO and ΔPC-AML1-ETO were in vitro translated, fractionated by gel filtration chromatography (Superose 6, SMART system, Pharmacia Biotech) and analysed by SDS PAGE followed by autoradiography. (C-D) Interaction of AML1-ETO with NCoR and DNA. (C) AML 1-ETO and ΔPC-AML 1-ETO were in vitro translated and then incubated with GST -NCoR (RDIII) or GST beads (C) as control in pull-down assays. Input lanes (I) represent 100% of the total. (D) extracts from AML 1-ETO U937 cells were incubated with biotinylated oligos containing a specific AML 1 binding site or an unrelated sequence (Ctrl), and then pulled-down with streptavidin-agarose beads (left panel). High-salt eluted material was subjected to gel filtration chromatography, to verify that DNA-bound AML1-ETO was still present as HMW complexes (right panel). (E) Transcriptional repression by AML 1-ETO. C33A cells were transiently transfected with the MDR1-luc reporter and the indicated expression vectors. Luciferase activity was determined as described in the legend to FIG. 2. (F) Analysis of myeloid differentiation. Lin-cells transduced with the indicated retroviral vectors were sorted and then treated as described in the legend to FIG. 5.

FIG. 8. Fusion of the coiled coil of PML to a transcription factor leads to the formation of high molecular weight complexes, enhanced recruitment of NCoR and enhanced transcriptional repression. (A) Thyroid receptor (TR) or a chimeric thyroid receptor fused C-terminally to the coiled coil of PML (CC-TR) were analyzed by gel filtration chromatography after labeling of transiently transfected COS-1 cells with iodinated thyroid hormone. (B) Increasing amounts of GST-NCoR (from 150 ng to 10 μg) coupled to agarose beads were incubated with the indicated in vitro translated, ³⁵-S labelled proteins. On the left, a schematic representation of the in vitro translated products (CC, coiled coil). (C) Transcriptional repression by TR and CC-TR. HeLa cells were co-transfected with the TRE-G5-TATA reporter in the absence (control) or presence of increasing amounts (50, 100, 250, 1000 ng) of the indicated expression vectors and then harvested 48 hours after transfection.

FIG. 9 shows example of sequences of coiled coil regions from additional proteins similar to PML in their primary sequence.

FIG. 10 shows the results of an experiment performed in U937 cells expressing PML-RAR under the control of an inducible promoter (Grignani et al., 1998). The cells were either uninduced (ctr column), or induced to express PML-RAR (PML-RAR column). Cells were transduced with retroviral vectors encoding the coiled coil of PML (RBCC) and GFP as a marker, or with retroviral vectors encoding GFP alone as a control (control). In control infections (gated in the cytofluorimetric analysis based on the positivity for the GFP marker), induction of PML-RAR strongly inhibited differentiation by treatment of the cells with vitamin D and TGFβ (as assessed by percentage of cells expressing the differentiation marker CD14)—from >90% to about 30% of differentiated, CD14+ cells-. In cells expressing the coiled coil of PML (gated in the cytofluorimetric analysis based on the positivity for the GFP marker), PML-RAR was unable to block efficiently differentiation (from 90% to 70%). Levels of expression of PML-RAR were unchanged by expressing the coiled coil region of PML. Notably, the inventors did not notice appreciable toxicity by expression of the coiled coil region of PML in the uninduced cells.

FIG. 11 shows the result of investigations into the capacity of coiled coil-mediated oligomers to inactivate cellular proteins.

A: p53 (lane 1) and CC-p53 (lane 2) were in vitro translated singly, or co-translated (lane 3). Samples were then immunoprecipitated with antibodies against the coiled coil region of PML (lane 4, from co-translated CC-p53 and p53; and lane 5, from p53 only), or with an anti-p53 specific antibody (lane 6, from p53 only).

B: Size exclusion chromatography (SEC) analysis of extracts from 293 cells transiently transfected with expression vectors for p53, CC-p53, or both vectors. Whole-cell lysates were prepared and subjected to SEC as described in the Materials and Methods section. Fractions were analysed by Western blotting using an anti-p53 antibody. Fraction number is indicated at the top of each lane.

C: p53 null murine embryonic fibroblasts (MEFs) were transiently transfected with a luciferase-based reporter vector for p53 transcriptional activity. This vector contains multimerized p53 response elements in front of a minimal promoter and of the reporter gene. The expression vectors indicated in the figure were co-transfected with the reporter and a β-galactosidase expression vector, used to normalize for transfection efficiency. Twenty-four hours after transfection, cells were collected and analyzed for reporter activity.

D: NIH 3T3 cells were transiently transfected with either an expression vector for a GFP-p53 fusion protein (A: left panel, GFP-p53; right panel: DAPI staining), or for CC-p53 (B: left panel, staining with an anti-coiled coil antibody; right panel: DAPI staining). In panels In parallel experiments, p53 was transfected in place of the GFP-p53 chimeric protein, with identical results (data not shown). In panels C-D-E, cells were co-transfected with CC-p53 and GFP-p53expression vectors (C, CC-p53; D, GFP-p53; E, merge).

E: SAOS cells (p53 null) were transfected with either a control vector (empty vector), or for vectors encoding p53, CC-p53, or both (1:1 ratio). The vectors contain a G418 resistance marker. Forty-eight hours after transfection, cells were split, and plated in medium containing G418 to select for transfected cells. Ten-twelve days after plating, G418-resistant colonies were counted.

### DETAILED DESCRIPTION

The Abnormal Recruitment of NCoR by PML. RAR is Caused by the Coiled Coil Region of PML

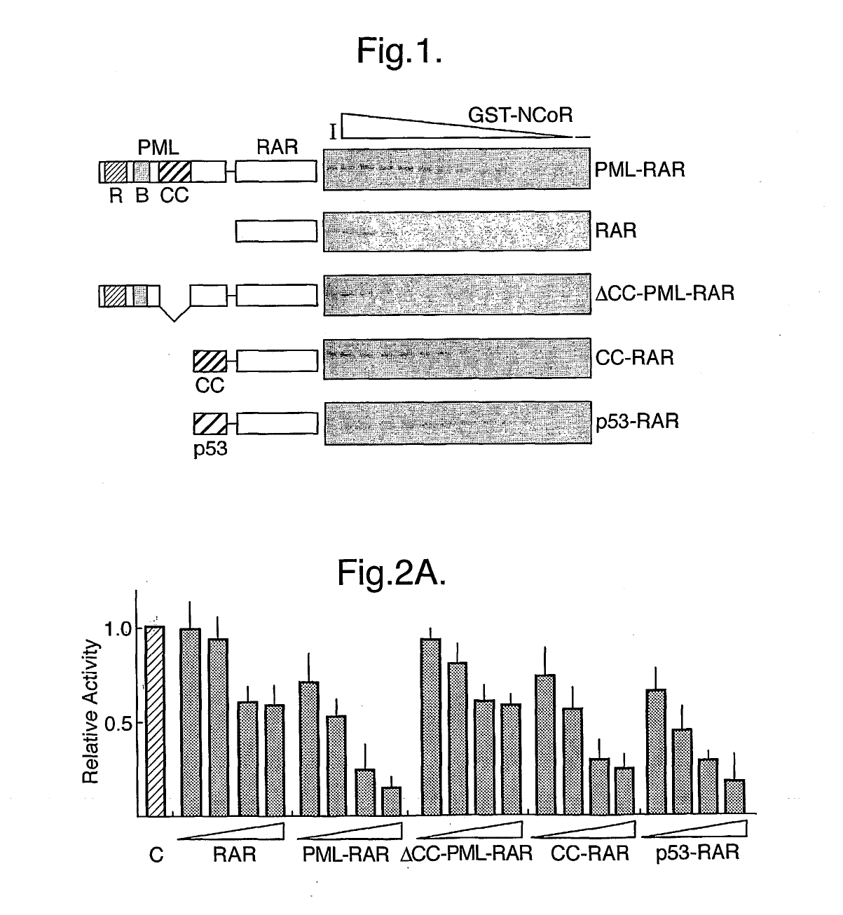

Recruitment of the NCoR-HDAC complex is crucial for the transforming potential of PML-RAR. At low concentrations of RA (1-100 nM), the stability of the PML-RAR-NCoR complex is higher than that of the RAR-NCoR complex (Grignani et al., 1998; He et al., 1998; Lin et al., 1998). Since PML does not interact directly with NCoR (Grignani et al., 1998), the present inventors investigated whether fusion to PML affected the stability of unliganded RAR for NCoR. Pull-down assays were performed by incubation of in vitro translated, ³⁵S labelled PML-RAR or RAR with GST-NCoR coupled to agarose beads. PML-RAR bound specifically to the beads even at the lowest amounts of GST-NCoR tested, whereas at least 15-30 fold higher amounts of GST-NCoR were required to obtain significant levels of RAR binding (FIG. 1). The inventors then mapped the region(s) in the fusion protein responsible for the enhanced stability of the NCoR interaction: deletion of the PML coiled coil region (ΔCC-PML-RAR) caused a dramatic decrease in the amount of bound protein, giving a pattern of binding essentially identical to wild-type RAR (FIG. 1). Deletion of other regions of PML (RING, B1 and B2 boxes) did not affect significantly the association of PML-RAR with NCoR (data not shown). Conversely, fusion of the coiled coil region of PML to RAR (CC-RAR) resulted in a chimeric protein with the same characteristics of binding as PML-RAR (FIG. 1). These results show that PML-RAR binds NCoR with higher apparent affinity than RAR and that the structural determinant for this altered association is the coiled coil region of PML.

The Coiled Coil Region of PML Determines the Altered Transcriptional Properties of PML-RAR

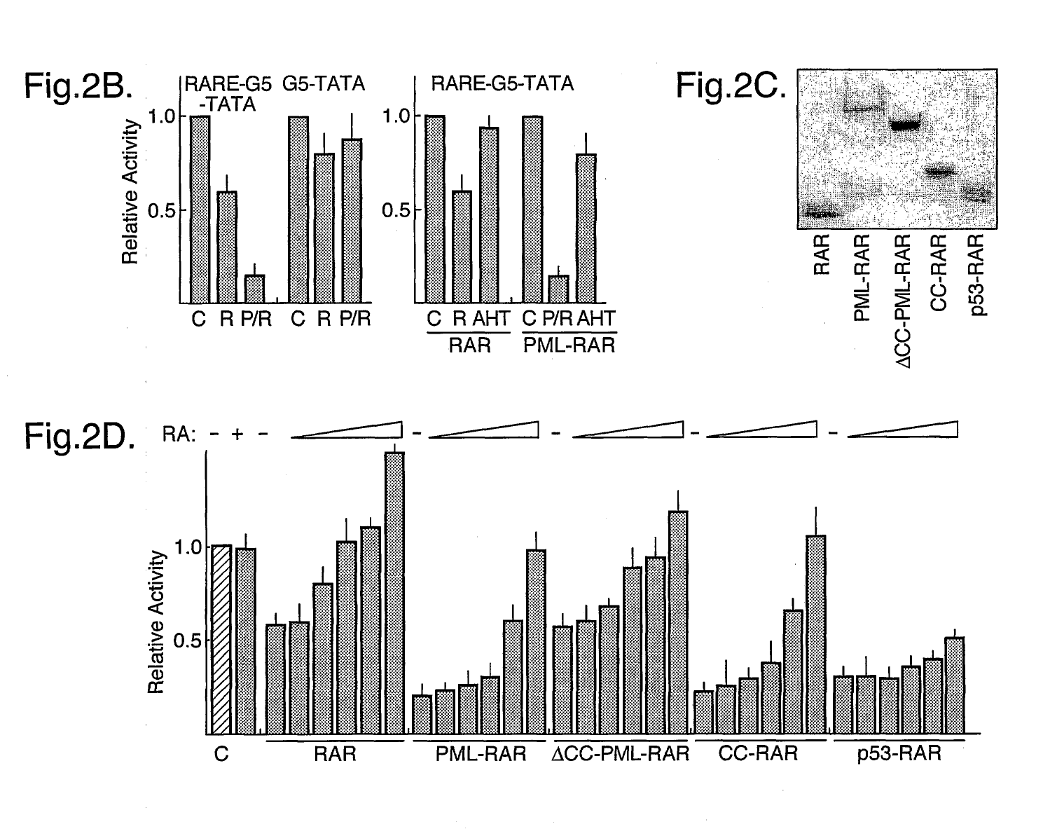

The enhanced binding of PML-RAR to NcoR suggests that PML-RAR might act as a more potent transcriptional repressor than RAR. To test this hypothesis, the inventors devised an artificial, reporter system to measure transcriptional repression by RAR and chimeric proteins. The RARE-G5-TATA reporter construct has five GAL4 response elements fused to a minimal promoter region: upstream of the GAL4 sites, a RA responsive element (RARE) allows binding of RAR (or fusion proteins). Transient transfection of this reporter in HeLa cells yielded low activity, while co-transfection with an expression vector for the GAL4-VP16 activator resulted in a strong response (10-13 fold induction; not shown). RAR over-expression led to decrease of GAL4-VP16 activation, with 30-40% repression observed with the maximal amount of co-transfected RAR expression vector (1 μg, FIG. 2A), while it exerted no effect on the GAL4-VP16-mediated activation of a cognate promoter lacking the RARE (FIG. 2B). A RAR construct carrying the AHT mutation, which abrogates NCoR binding (Horlein et al., 1995), was unable to prevent activation of the RARE-G5-TATA promoter by GAL4-VP16 (FIG. 2B).

Next, the inventors tested the transcriptional regulatory functions of PML-RAR (or derivatives). Transfection of expression vectors for RAR, PML-RAR or the various mutants yielded comparable levels of protein expression (FIG. 2C). Whereas it was necessary to transfect at least 250 ng of the RAR expression vector to measure significant transcriptional repression, transfection of 50 ng of the PML-RAR expression vector resulted in relevant repression (30-40%). At the maximal concentration tested (1 μg), the inventors observed 80-90% repression with PML-RAR and 30-40% with RAR (FIG. 2A). Co-transfections of the PML-RAR expression vector with a RARE-less reporter abrogated transcriptional repression, and the PML-RAR AHT expression vector had no effect on the activation of the RARE-G5-TATA promoter by GAL4-VP16, showing that—as for RAR—transcriptional repression by PML-RAR requires DNA binding and is dependent on the recruitment of NCoR (FIG. 2B). The ΔCC-PML-RAR construct was identical to RAR in its capacity to weakly repress GAL4-VP16 driven transcription, whereas CC-RAR repressed GAL4-VP16 activity as strongly as PML-RAR (FIG. 2A).

The present inventors have previously shown that higher concentrations of RA are required to dissociate PML-RAR (as opposed to RAR) from NCoR (Grignani et al., 1998). To address the role of the coiled coil region on the stability of the association with NCoR in the presence of RA, they performed the same transfection assays in the presence of RA (1 nM-10 μM). Near-physiological RA concentrations (up to 100 nM) reverted RAR and ΔCC-PML-RAR repression, whereas much higher concentrations (1-10 μM) were required to revert repression by PML-RAR and CC-RAR (FIG. 2C).

These results show that PML-RAR is a stronger transcriptional repressor than RAR, and its activity and altered RA sensitivity require the PML coiled coil region.

The Coiled Coil Region of PML is Responsible for the Oligomeric PML-RAR Complexes

Integrity of the coiled coil region is required for the biological properties of PML-RAR (Grignani et al., 1996). The PML coiled coil region is also responsible for the appearance of PML-RAR within high molecular weight (HMW) complexes, as shown by gel filtration analysis of nuclear extracts from PML-RAR expressing cells (Grignani and al., 1999; Nervi et al., 1992). Fusion of the PML coiled coil region to RAR may change the composition of RAR-associated factors leading to enhanced recruitment of NCoR, transcriptional deregulation, and oncogenic activity. To investigate this possibility, the present inventors analysed the molecular identity of these complexes.

The elution profile of PML-RAR was previously analysed by incubation of the extracts with tritiated RA and measurement of the radioactivity of RA-bound polypeptides (Benedetti et al., 1997; Nervi et al., 1992; Grignani et al., 1999). To eliminate the possibility that RA would shift the identity of PML-RAR associated factors, the inventors decided to analyse unliganded complexes in fractions eluted from a gel filtration column by Western blotting, using anti-RAR antibodies.

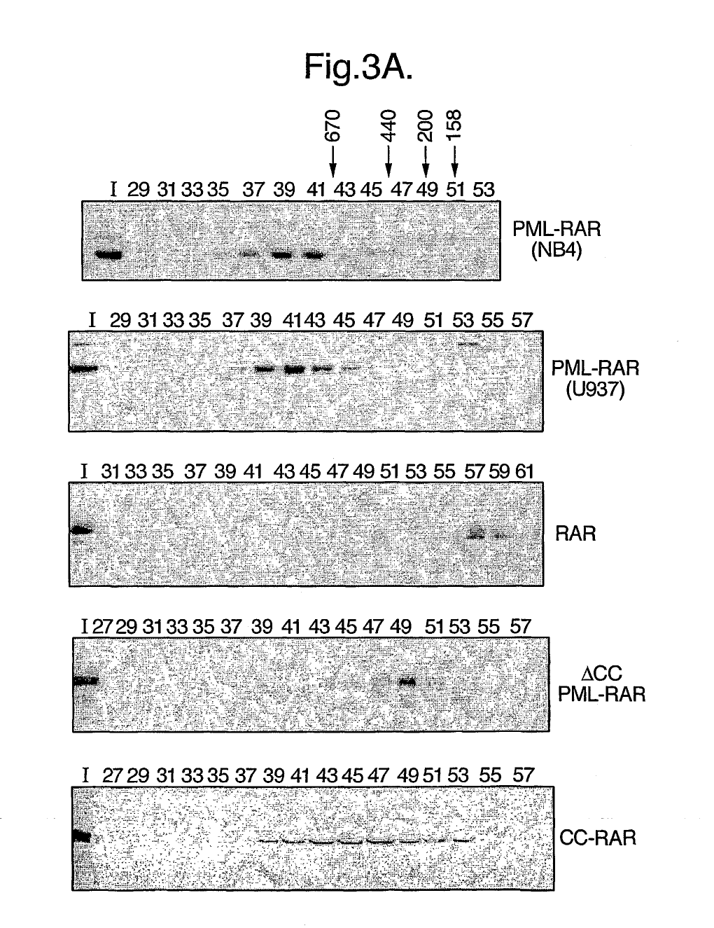

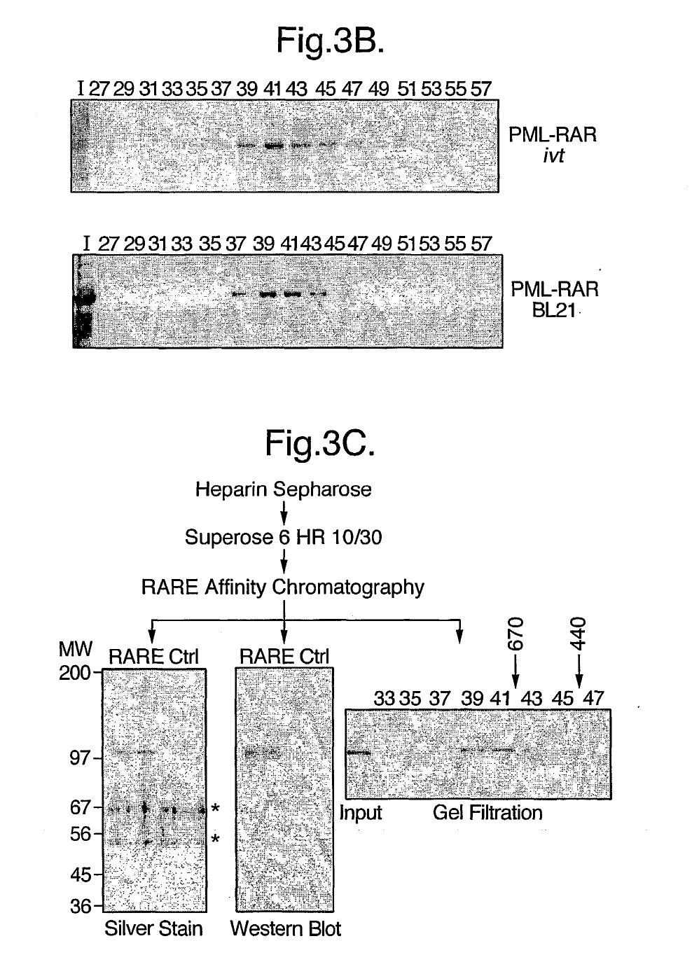

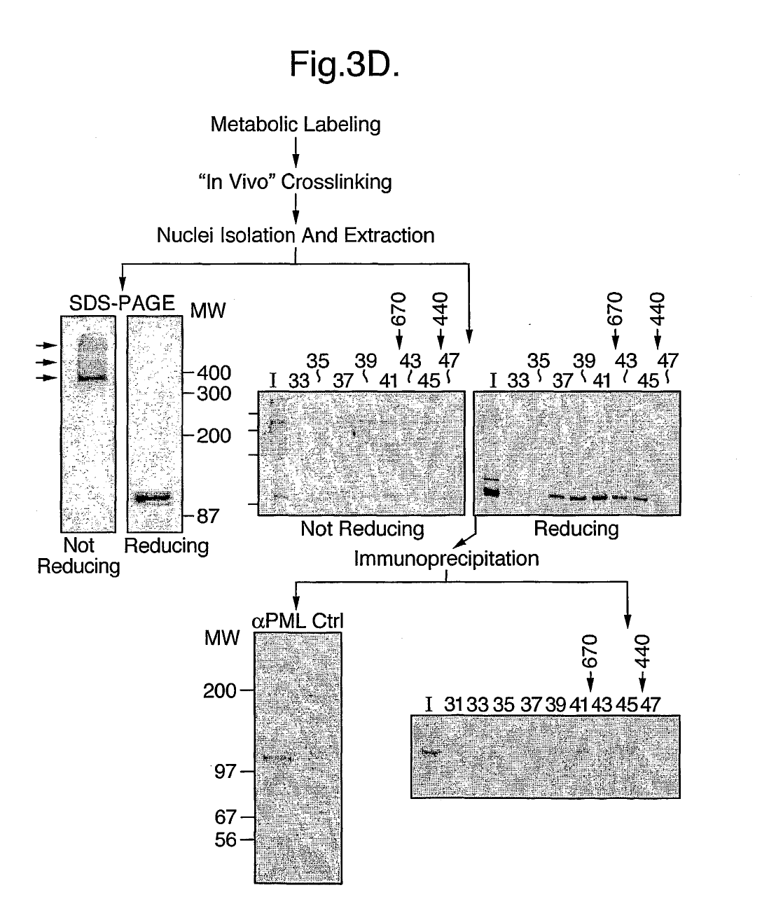

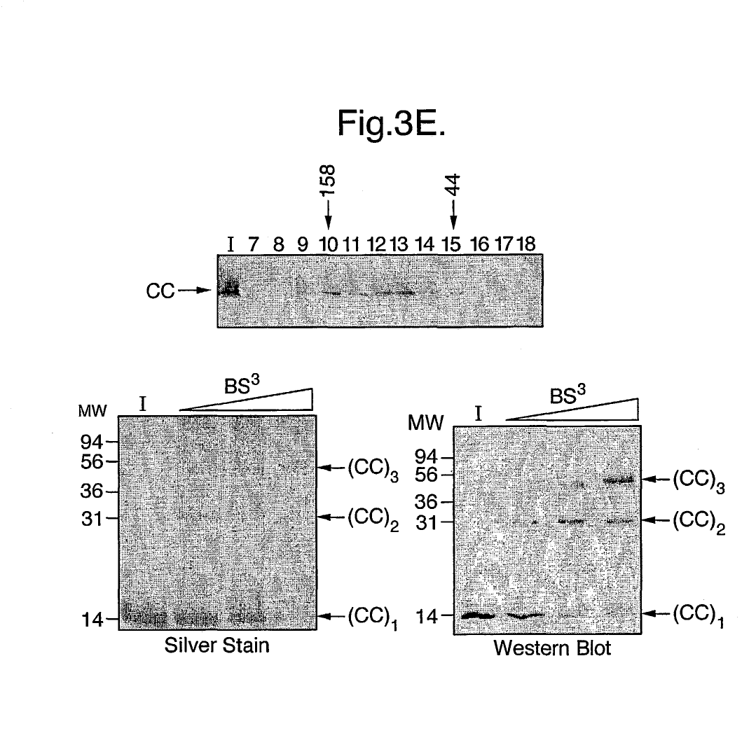

In extracts from all PML-RAR expressing cells examined (fresh APL blasts, and the promyelocytic NB4 cell line), unliganded PML-RAR was present in gel filtration fractions peaking with an apparent molecular weight of about 700 kDa (FIG. 3A and data not shown). This elution volume is identical to that previously observed using titrated RA (Nervi et al., 1992). In contrast, RAR always eluted as a monomeric species (FIG. 3A). Deletion of the PML coiled coil region shifted the elution volume of the PML-RAR complexes to regions corresponding to lower molecular weight (mono- or dimeric) species, while CC-RAR was found in HMW complexes, confirming that the coiled coil domain of PML is necessary and sufficient for the formation of HMW complexes (FIG. 3A).

To evaluate whether formation of HMW complexes is an intrinsic property of the fusion protein, the inventors analysed in vitro translated and bacterially expressed PML-RAR. They expressed and purified PML-RAR in bacteria as an MBP-PML-RAR fusion protein and then removed the MBP moiety by factor Xa cleavage. Gel filtration analysis revealed that in vitro translated and bacterially expressed PML-RAR was still found in HMW complexes (FIG. 3B), suggesting that the PML-RAR nuclear complexes consist of oligomeric PML-RAR. To test this hypothesis, the inventors purified PML-RAR from nuclear extracts. Our purification scheme includes several chromatographic steps: heparin-Sepharose, Superose 6, and, as final step, DNA-affinity on a biotinylated RARE coupled to streptavidin-agarose beads. Only one specific 120 kDa band (corresponding to PML-RAR, as shown by parallel Western blot analysis) was observed after silver stain analysis of the purified material (FIG. 3C). Gel filtration analysis revealed that the eluted PML-RAR was still present in HMW complexes (FIG. 3C), indicating that the HMW complex isolated from nuclear extracts reflect PML-RAR in oligomeric complexes.

To investigate whether the PML-RAR oligomeric complexes exist in vivo, the inventors used the cell membrane-permeable, reversible cross-linking agent DTBP in in vivo cross-linking experiments. Lysates were prepared from in vivo cross-linked, metabolically labelled cells. SDS-PAGE analysis of the cross-linked material was performed under non-reducing conditions (to preserve the cross-linking): in addition to the 120 kDa PML-RAR band, a more intense, >350 kDa band, and a series of less well resolved bands of higher MW (FIG. 3D), were recognised in Western blot using anti-RAR antibodies. These last bands were absent in gels run in reducing conditions (to revert the cross-link) and from non cross-linked material (FIG. 3D and data not shown), and represented the cross-linked material. In parallel, the same nuclear extracts were subjected to gel filtration: Western blot analysis from gels run in reducing or non-reducing conditions—of the fractions revealed that the cross-linked material was still present in HMW complexes (FIG. 3D). The fractions corresponding to HMW complexes were then pooled and immunoprecipitated using an anti-PML specific antibody. The immunoprecipitated complex contained exclusively one 120 kD ³⁵-S labelled polypeptide (FIG. 3D), that was recognised by anti PML and anti-RAR antibodies (not shown), co-migrated with PML-RAR in SDS-PAGE and was absent in immunoprecipitates from samples prepared in identical conditions from control cells (not shown). The inventors conclude that the labelled polypeptide represents PML-RAR, and that no other cellular proteins are stoichiometrically cross-linked under these conditions. The inventors eluted the immunocomplexes by SDS 1% and then subjected the eluted PML-RAR to a new round of gel-filtration: interestingly, they found that PML-RAR was still present in HMW complexes (FIG. 3D). The same complexes were not recoverable from non cross-linked material immunoprecipitated from HMW complexes and then used as a control (data not shown). Together, these results indicate that the oligomeric status of PML-RAR pre-exist in vivo prior to cell lysis, and represents the natural form of organisation of PML-RAR within the cell nucleus.

Estimation of the molecular mass of the PML-RAR oligomers by size fractionation has intrinsic limitations. Indeed, the coiled coil region of PML (although relatively small—approximately 100 residues—compared to the total size of the protein—about 1,000 residues—) may influence the shape of PML-RAR (Hirano and Mitchison, 1994). By use of a complementary approach (centrifugation through a sucrose gradient), unliganded PML-RAR sedimented at a position consistent with a MW 700 kDa (not shown). Calculation of the molecular mass of the oligomeric complex based on Stokes radius (from gel filtration assays) and the sedimentation coefficient (from sucrose gradients), the inventors could obtain an estimation of the molecular mass of PML-RAR oligomers without assumptions about the shape of the protein (Siegel and Monty, 1966): from those values, the inventors estimated a molecular mass of 650-700 kDa, which is consistent with the formation of a PML-RAR hexamer.

There are no known cases, however, of coiled coil domains mediating the formation of oligomeric hexamers (Lupas, 1996). To address this issue, we investigated the molecular properties of the isolated coiled coil domain of PML. The 14 kDa coiled coil domain (obtained by site-specific cleavage of a purified GST-fusion protein) eluted as a defined HMW peak (60-150 kDa), confirming its capacity to oligomerize (FIG. 3E, upper panel). To assess its oligomerization number, we subjected the isolated coiled coil to in vitro cross-linking studies. Treatment with two different cross-linkers (DT8P or BS³; see methods) resulted in the formation of higher MW bands, corresponding to di- and tri-meric species of the coiled coil domain (FIG. 3E and data not shown). Immunoblot experiments confirmed that the higher MW bands corresponded to cross-linked coiled coil (FIG. 3E). Identical results were obtained by cross-linking experiments performed on the purified coiled coil domain obtained from the gel filtration columns (data not shown). It appears, therefore, that the coiled coil domain can be isolated from bacteria as a trimeric complex, raising the question of its relationship with the observed PML-RAR hexameric complex. Two possible explanations can be envisaged: I) the oligomeric PML-RAR is a trimeric complex with different migration properties with respect to the globular protein used as MW markers (Hirano and Mitchison, 1994; Lupas, 1996); ii) the oligomeric PML-RAR complex is a trimer-trimer complex, due to additional protein-protein interactions mediated by other domains of PML (RING, B-boxes) or RAR. In support of this latter hypothesis, we noted that ΔCC-PML-RAR eluted as mono- and dimeric species (FIG. 3A), suggesting the presence of additional, coiled coil-independent interactions among PML-RAR molecules. A two-step oligomerization mechanism has been recently described for tenascin-C, where the formation of a parallel three-stranded coiled coil stabilizes the connection of two triplets to a hexamer through an accessory interaction domain (Kammerer et al., 1998).

PML-RAR Oligomers Recruit NCoR and RXR to RA—Responsive Elements

Recruitment of NCoR and RXR, as well as specific binding to DNA, are critical for the oncogenicity of PML-RAR (Minucci and Pelicci, 1999). Therefore, the inventors investigated whether the oligomeric form of PML-RAR can associate with NCoR, RXR and specific DNA responsive elements.

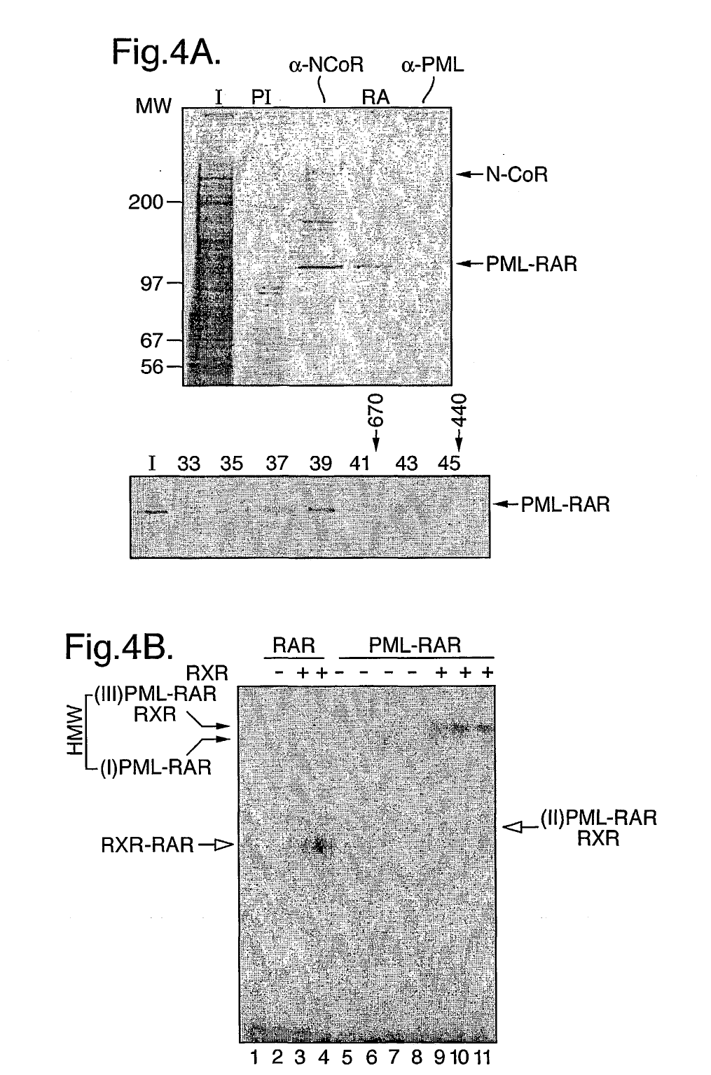

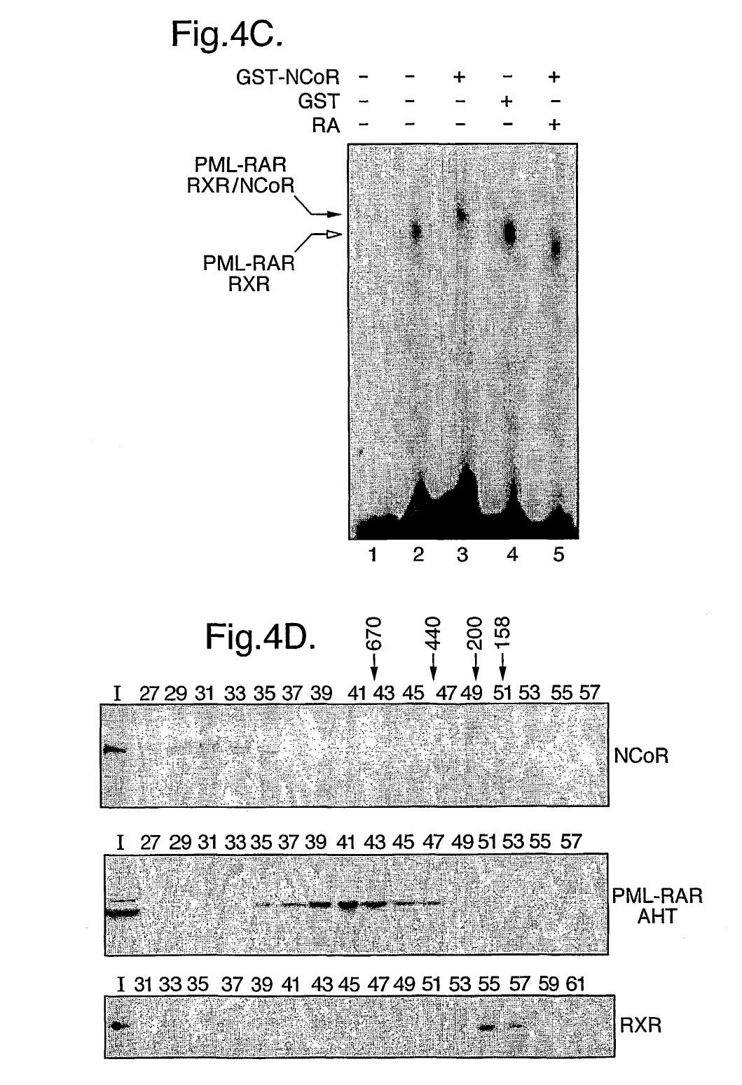

To investigate the association with NCoR, we analyzed anti-NCoR immunoprecipitates from metabolically labeled nuclear extracts of cells expressing PML-RAR. An approximately 120 kDa protein co-precipitated with NCoR (FIG. 4A). We identify the 120 kDA, NCoR-associated factor as PML-RAR, based on the following criteria: I) it co-migrated with PML-RAR (as determined by parallel immunoprecipitation of the same nuclear extracts with anti-PML or anti-RAR antibodies: FIG. 4A and data not shown); ii) it was not present in the immunoprecipitates from cells not expressing PML-RAR (data not shown); and iii) it could be specifically dissociated from the NCoR immunoprecipitated complexes by incubation with RA (FIG. 4A). To determine whether the oligomeric form of PML-RAR was able to associate with NCoR in vivo, we performed gel filtration analysis of the RA-eluted fraction from anti-NCoR immunoprecipitates. As shown in FIG. 4A, lower panel, PML-RAR was recovered in HMW complexes, demonstrating the existence of an oligomeric PML-RAR/NCoR complex in vivo.

To establish whether PML-RAR oligomers bind DNA specifically, the inventors expressed PML-RAR (or RAR) into Xenopus oocytes. This system has been widely used to study the transcriptional regulatory functions of nuclear receptors (including RARs: Wong et al., 1998; Minucci et al., 1998) and contains low to undetectable levels of endogenous receptors (unlike mammal cells), thus allowing unambiguous evaluation of the DNA binding properties of exogenous receptors (Wong et al., 1996; Minucci et al., 1998). Since RAR and PML-RAR have been previously shown to require RXR for high efficiency binding to DNA responsive elements, in some experiments we coinjected mRNAs for RXR to express RXR/RAR or PML-RAR/RXR complexes. Extracts from injected oocytes were tested in mobility shift assays, using the RA responsive element (RARE) from the RARβB2 promoter as a probe. As previously shown, injection of mRNA for RAR resulted in no detectable binding complex, whereas coinjection of mRNA for RXR caused the formation of a strong heterodimeric RXR/RAR DNA binding complex (FIG. 4B; Minucci et al., 1998). Expression of PML-RAR resulted in the formation of a complex (complex I) which migrated considerably more slowly than the heterodimeric RXR-RAR complex (FIG. 4B). This complex was specific, since it was competed by an excess of cold RARE (but not by an unrelated oligonucleotide: data not shown). To establish conclusively whether the PML-RAR/DNA slowly migrating complex corresponds to HMW PML-RAR bound to DNA, the present inventors performed size exclusion chromatography analysis of the radioactively labelled RARE/PML-RAR complex in the same buffer conditions as the mobility shift assays. In the absence of cell extracts, or after incubation with control extracts lacking PML-RAR, the RARE was completely absent in fractions corresponding to a predicted MW of 30-60 kDa, consistent with its length and size (37 double-stranded oligonucleotide). In PML-RAR containing extracts, however, a new peak of radioactive RARE was observed, precisely co-fractionating with PML-RAR and corresponding to an apparent MW>670 kDa (data not shown). Parallel analysis of the same samples in mobility shift assays revealed the appearance of the PML-RAR/DNA slowly migrating complex, showing that this complex indeed represented the binding of HMW PML-RAR to the DNA. As for RAR, coexpression of RXR resulted into enhanced binding to DNA (FIG. 4B). Notably, two novel complexes were formed in this case: I) a low abundance complex (complex II), that migrated slightly slower than the RXR/RAR heterodimer and that we interpret as the heterodimer formed by (monomeric) PML-RAR and RXR; ii) a highly abundant complex (complex III), that migrated much more slowly than complex II and slightly slower than complex I and that we interpret as the oligomeric PML-RAR/RXR DNA binding complex (FIG. 4B).

Further mobility shift assays were performed to determine whether NCoR might be recruited to the PML-RAR/RXR/DNA complex. We used agarose as a solid matrix for the electrophoretic runs in this case, to allow better resolution of very high molecular weight complexes (in the range of 500 kDa-1 MDa). The oligomeric PML-RAR/RXR/DNA complex was super-shifted by the addition of recombinant GST-NCoR(but not control GST: FIG. 4C). RA addition caused the disappearance of the supershift and the formation of an oligomeric PML-RAR/RXR/DNA complex that migrated slightly faster than the complex observed in the absence of RA (FIG. 4C, lane 2 against lane 5). It has been previously observed that ligand binding to RAR and other nuclear receptors results in a faster migration of the receptor/DNA binding complex, likely as a result of a conformational change in the receptor ligand binding domain induced by RA (reviewed in Chambon, 1996). These results demonstrate that PML-RAR oligomers bind DNA, that DNA binding is enhanced by RXR recruitment, and that NCoR may be recruited to the oligomeric PML-RAR/RXR/DNA complex. The association of PML-RAR oligomers with NCoR and RXR does not contradict the inventors' finding that PML-RAR HMW complexes originate exclusively from oligomerization of the fusion protein, in the absence of other cellular proteins interacting stoichiometrically (FIG. 3). In fact, neither the NCoR/HDAC complex nor RXR co-fractionated with PML-RAR in gel filtration of lysates from PML-RAR expressing cells, and the PML-RAR AHT mutant, that is unable to recruit the NCoR/HDAC complex, has an elution profile coinciding with PML-RAR (FIG. 4D). It appears, therefore, that PML-RAR can be isolated as tightly interacting, self-associating oligomeric complexes which represent the “core” complex responsible for the interactions (at lower affinity and/or stoichiometry) with other factors, such as nuclear corepressors and RXR.

Fusion with a Heterologous Oligomerization Domain Increases NCoR Binding and the Transcriptional Repressive Activity of RAR and Activates its Leukaemogenetic Potential

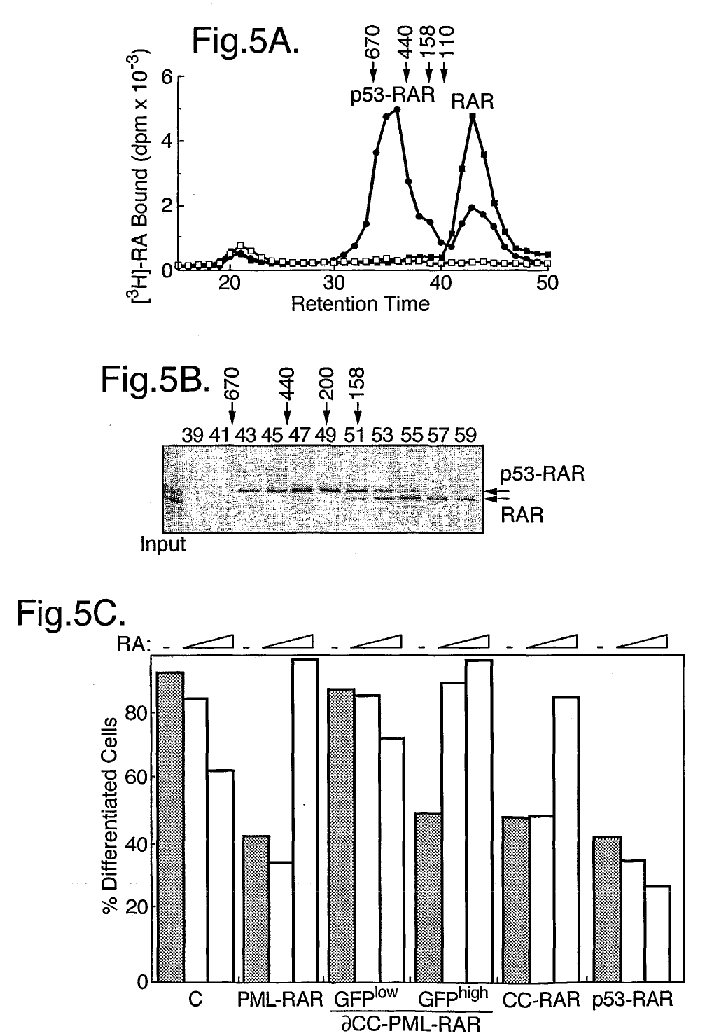

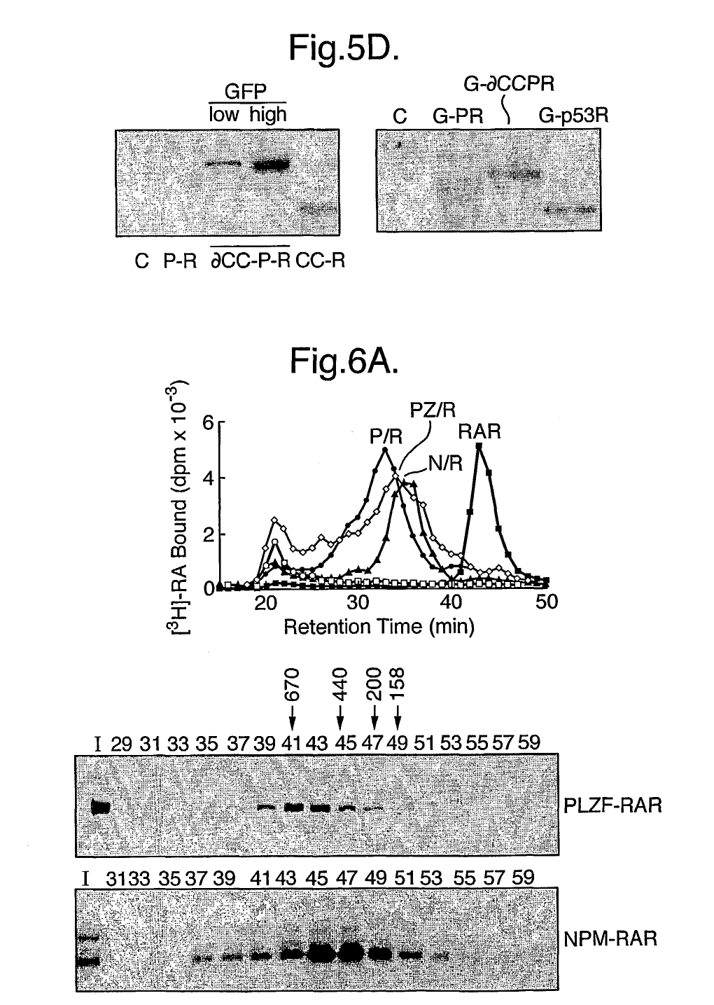

The inventors' results point to a critical role for the coiled coil region of PML in mediating oligomerization, which represents the structural determinant for the aberrant interaction with the NCoR/HDAC complex and for leukemogenetic activity of the fusion protein. To determine whether the oligomerization per se is the critical function, or if other properties of the PML coiled coil region play a role, the inventors evaluated the effects of a heterologous oligomerization domain on the transcriptional and biological properties of RAR. To this end, they fused RAR C-terminally to the tetramerization domain present in p53, a well-studied self-association module that allows tetramerization of heterologous proteins(Chen et al., 1998; Clore et al., 1994). COS-1 cells were transfected with a p53-RAR expression vector, and nuclear extracts were labelled with tritiated RA. RA binding capacity was analysed by gel filtration chromatography: the profile obtained showed that p53-RAR formed HMW complexes (FIG. 5A). The fusion protein was then in vitro translated and analysed by gel filtration chromatography: compared to RAR, which elutes as a monomer, p53-RAR was found in HMW complexes (FIG. 5B). These results confirm that the oligomerization domain of p53 is able to impose the self-association of RAR, allowing us to evaluate the biological activity of RAR oligomers that do not contain PML sequences..

The inventors performed pull-down assays by incubation of in vitro translated, ³⁵S labeled p53-RAR with increasing concentrations of GST-NCoR. Similarly to PML-RAR, p53-RAR bound NCoR even at the lowest amounts of GST-NCoR tested (FIG. 1). The inventors then measured the capacity of p53-RAR to repress GAL4-VP16 driven transcription: as shown in FIG. 2A, p53-RAR repressed GAL4-VP16 activity as strongly as PML-RAR and the CC-RAR mutant, and was a more potent transcriptional repressor than the natural RAR.

The capacity of p53-RAR to block differentiation was evaluated using primary hematopoietic precursors purified from murine bone marrow on the basis of the absence of surface differentiation antigens (lin-see methods). Lin-cells were transduced using retroviral constructs encoding for PML-RAR (or derivatives) and GFP as a marker: Cells transduced with the control retroviral vector-expressing GFP only—behaved identically to uninfected cells (data not shown). GFP-positive cells were sorted and seeded in methylcellulose plates containing a cytokine cocktail (including G-CSF and GM-CSF), to allow terminal myeloid differentiation. After 8-10 days, colonies were pooled and analysed for the expression of myeloid differentiation markers (Mac-1 and GR-1). The results for Mac-1 are presented in FIG. 5C: similar results were obtained for GR-1 (data not shown). Compared with control cells, PML-RAR expressing cells showed a strong reduction in their capacity to differentiate (FIG. 5C). Cells expressing ΔCC-PML-RAR showed—upon sorting—much higher levels of expressed protein compared to PML-RAR, as judged by Western blot (data not shown and FIG. 5D). It has been shown that high levels of RAR lead to a differentiation block. This is likely to be owing to sequestering of available RXR (Du et al., 1999; Grignani et al., 1996). For this reason, the inventors sorted the ΔCC-PML-RAR GFP+ cells into two populations, according to their mean fluorescence levels: correspondingly, Western blot analysis revealed that GFP^{low} cells expressed lower levels of ΔCC-PML-RAR than the GFP^{high} cells, although the level of the chimeric protein was at least 2-3 fold higher than PML-RAR (FIG. 5D). ΔCC-PML-RAR had essentially no effects on differentiation of the GFP^{low} infected cells, whereas at higher levels it induced a consistent differentiation block (>30%, compared to about 50% block for PML-RAR). Lin-cells were then infected with a retroviral construct encoding p53-RAR as a GFP-fusion protein. Infected cells were sorted and plated in methylcellulose differentiation medium as described before. The inventors observed a strong decrease in the number of Mac1+ and GR1+ cells in the p53-RAR infected sample compared to control cells, similarly to what was observed upon PML-RAR or GFP-PML-RAR expression (FIG. 5C and data not shown). At comparable levels of expression, a GFP-ΔCC-PML-RAR construct had no effects on the differentiation of transduced cells (data not shown). Taken together, these observations demonstrate that addition of an oligomerization domain to RAR is sufficient to obtain a fusion protein with full transforming potential.

The present inventors then compared the capacity of PML-RAR, CC-RAR, and p53-RAR to mount a RA-response in transduced murine primary haemopoietic precursors, measuring the capacity of RA to relieve the differentiation block due to expression of RAR-fusion proteins. The differentiation block by PML-RAR and CC-RAR was relieved exclusively at high concentrations of RA (1 μM, FIG. 5C). In contrast, p53-RAR expressing cells were insensitive to RA treatment at all concentrations (FIG. 5C). Similar results were obtained in p53-RAR U937 cells, excluding the contribution of cell-type specific effects (data not shown). Accordingly, RA did not relieve transcriptional repression by p53RAR on a RARE-containing reporter, in contrast with PML-RAR and CC-RAR that were fully responsive at high RA concentrations (FIG. 2D). Use of other RAR ligands (9-cis-RA, TTNPB) resulted in identical results (data not shown). Scatchard analysis performed to measure the affinity of RA for p53-RAR yielded a calculated apparent equilibrium dissociation constant (Kd) of 4.0+/−0.42 nM (mean +/−SD, n=3), compared to 0.5 nM observed for RAR, PML-RAR and CC-RAR in parallel samples (data not shown). Thus, the affinity of RA for p53-RAR is lower than for PML-RAR. At the concentrations of RA used in the experiments above (1-10 μM; 4,000 higher than the measured Kd), however, this difference is unlikely to be significant. It appears, therefore, that RAR-fusion protein oligomers exert differential responses to RA on the basis of the identity of the oligomerization domain fused to RAR.

PML, PLZF and NPM Form HMW Complexes In Vivo and Induce Oligomerization of their Corresponding RAR Fusion Proteins

Other RAR fusion proteins (such as PLZF-RAR and NPM-RAR) are infrequently associated with the promyelocytic leukemia phenotype (Melnick and Licht, 1999). They share with PML-RAR the same portion of RAR and the ability to block differentiation, to recruit the NCoR-HDAC complex and to deregulate expression from RA-target genes (Minucci and Pelicci, 1999; Redner et al., 1999). Although analysis of PLZF and NPM sequences failed to identify putative coiled coils, both PLZF and NPM contain strong protein-protein interaction domains directing their self-association and retained within the corresponding fusion proteins (Ahmad et al., 1998; Chan and Chan, 1995). PLZF-RAR has been shown to form HMW nuclear complexes (Benedetti et al., 1997). The present inventors investigated whether the three RAR translocation partners PML, PLZF and NPM are able to form HMW nuclear complexes.

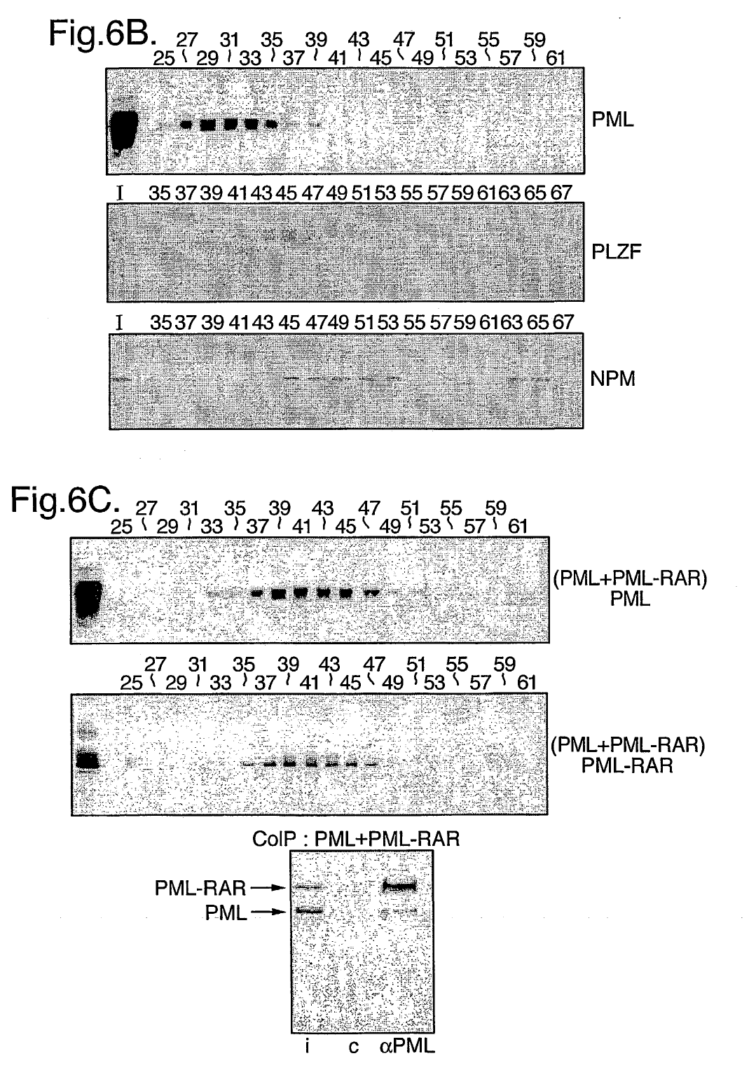

PML is tightly associated to the nuclear matrix, and was not extracted in the inventors' experimental conditions (Chang et al., 1995). Over expression of PML provokes a partial solubilization, and approximately 10-20% of the protein is then found in the nucleoplasmic fraction of nuclear extracts (data not shown). Gel filtration analysis of nuclear extracts from transfected HeLa cells revealed that PML is distributed in HMW complexes with an apparent MW ranging from >670 kDa to the void volume of the column (FIG. 6B). Identical results were obtained by expressing PML in bacteria, indicating that the HMW complexes correspond to oligomeric PML forms (data not shown). PLZF and NPM were also found in HMW complexes (FIG. 6B). PLZF peaked at an apparent MW>440 kDa, whereas NPM was found in two distinct pools, as a monomer (30% of total NPM) or in HMW complexes (ranging approximately from 200 to >400 kDa) consistent with an hexameric state, as previously described (Chan and Chan, 1995). Gel filtration analysis of NPM-RAR-expressing nuclear extracts labeled with tritiated RA showed distribution patterns similar to those of PML-RAR and PLZF-RAR, although it peaked with a slightly lower apparent MW (400 kDa), consistent with the lower MW of NPM-RAR compared to PML-RAR (60 kDa versus 120 kDa, FIG. 6A). It appears, therefore, that RAR-fusion protein all form HMW complexes in vivo through their corresponding PML, PLZF or NPM moieties.

The ability of the PML coiled coil region (unlike the oligomerization domain of p53) to restore the RA response suggests that it may recruit additional nuclear factors to PML-RAR oligomers. The inventors therefore looked at the possibility that PML itself might be recruited to PML-RAR complexes. Gel filtration chromatography of extracts from HeLa cells transiently transfected with PML and PML-RAR expression vectors showed co-fractionation of PML and PML-RAR (FIG. 6C). Analysis of anti-RAR immunoprecipitates from fractions containing PML and PML-RAR revealed the presence of PML, showing that PML can be recruited to PML-RAR HMW complexes (FIG. 6C). RA treatment did not modify PML-RAR complexes or affected PML recruitment to PML-RAR oligomers (data not shown): therefore, PML is a candidate PML-RAR co-factor in the RA-response of APL cells.

Oligomerization of the AML1/ETO Fusion Protein

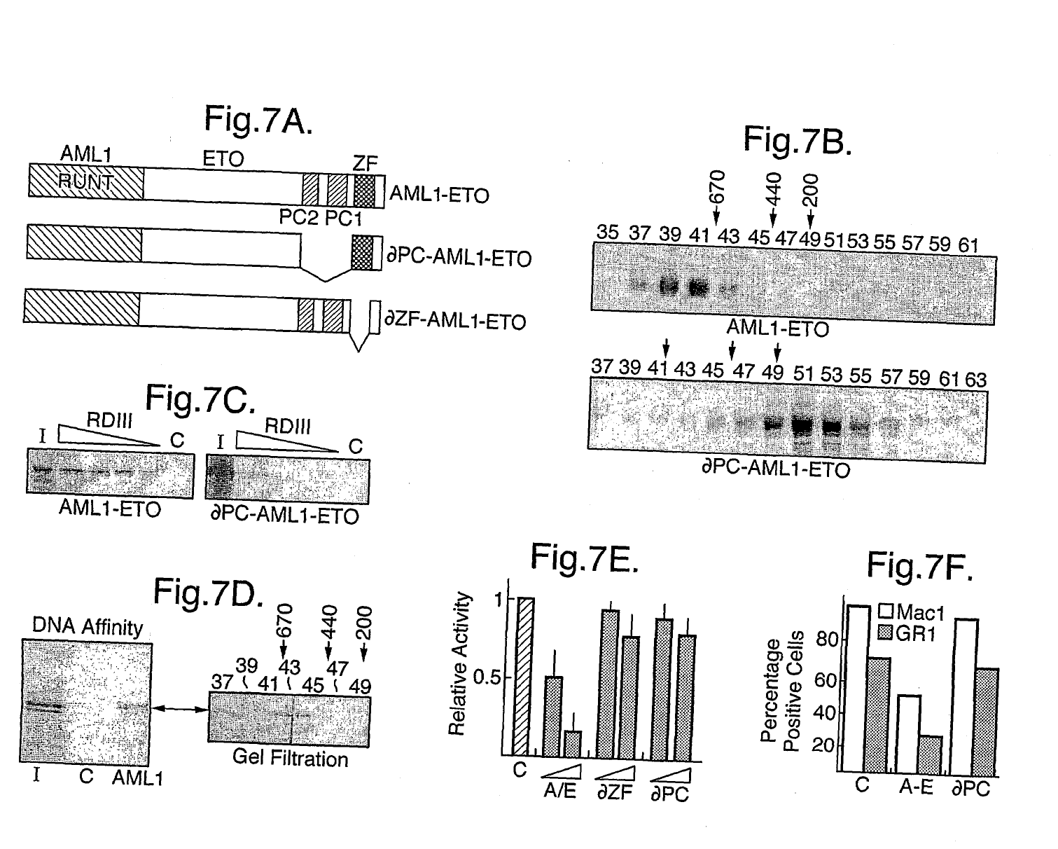

Mutation of the NCoR binding site impairs the biological activity of AML 1-ETO (Gelmetti et al., 1998). In this case, recruitment of NCoR-HDAC is mediated by ETO and might be sufficient to alter the function of AML 1. However, an AML1-HDAC1 fusion protein was unable to block hematopoietic differentiation (not shown), suggesting that recruitment of HDAC is not sufficient to activate the oncogenic potential of AML 1. Analysis of the ETO primary sequence revealed two putative protein-protein interaction domains: a coiled coil region (PC1, residues 444-492) and an amphipatic α-helix (PC2, residues 352-378; Lutterbach et al., 1998) (FIG. 7A). Since ETO has been shown previously to form HMW complexes (Lutterbach et al., 1998), the inventors performed gel filtration analysis of AML 1-ETO and of a defective mutant of the fusion protein lacking PC1-PC2 (ΔPC-AML1-ETO in FIG. 7A). AML 1-ETO was found within HMW fractions, while deletion of PC1 and PC2 regions shifted the fusion protein to lower molecular weight forms (FIG. 7B and data not shown). AML1a was found as monomeric species, confirming the requirement for ETO in the formation of HMW complexes by the fusion protein (data not shown). As a further characterisation of the HMW complexes, the inventors expressed and purified AML 1-ETO from bacteria as an MBP-AML 1-ETO fusion protein and then removed the MBP moiety by factor Xa cleavage. Gel filtration analysis revealed that bacterially expressed AML 1-ETO formed HMW complexes identically as the in vitro translated form, indicating its oligomeric state (data not shown). The inventors then investigated if the loss of the capacity to form HMW complexes correlated also with changes in the ability of AML 1-ETO to recruit NCoR and to repress transcription. The ETO interaction site for NCoR has been mapped in vitro at the two C-terminal zinc finger motifs (Gelmetti et al., 1998; Lutterbach et al., 1998). Deletion of the PC1 and PC2 motifs led to a strong decrease in the amount of fusion protein bound to GST-NCoR, despite the fact that the NcoR binding site is retained in the APC-AML1-ETO fusion protein (FIG. 7C). Since the PC motifs and NcoR do not interact in vitro (unpublished results), these data indicate that the PC motifs contributes to NCoR recruitment by AML1-ETO through their ability to mediate the formation of HMW complexes.

AML 1-ETO has been shown to bind DNA alone or as AML 1-ETO/CBFβ complexes (Meyers et al., 1995). Since the DNA binding complex is the effector of the leukemogenic effect of AML 1-ETO, the inventors analyzed whether HMW AML 1-ETO complexes are able to bind DNA. To this end, we partially purified AML 1-ETO from AML 1-ETO expressing cells by DNA affinity (using a specific AML 1 specific response element; see methods and FIG. 7D, left panel). Gel filtration of DNA-eluted material showed that AML 1-ETO can be recovered in its oligomeric form after DNA binding (FIG. 7D, right panel), suggesting that the oligomeric AML 1-ETO/DNA complex might recruit NcoR and efficiently repress transcription. Consistently, deletions of either the NCoR binding site (ΔZF-AML1-ETO) or the oligomerization regions (ΔPC-AML1-ETO) greatly impaired the capacity of the fusion protein to: I) repress transcription from a target promoter—MDR-1—in transient transfection assays (FIG. 7E and Lutterbach et al. , 1998); and ii) block differentiation of primary hemopoietic progenitors (FIG. 7F). Taken together, these results indicate that the efficient recruitment of the NCoR-HDAC complex by AML1-ETO is required to activate the oncogenic potential of AML1, and that this is achieved by the formation of AML1-ETO HMW complexes.

Fusion of the Coiled Coil Domain of PML to a Heterologous Transcription Factor Enhances its Functional Activity

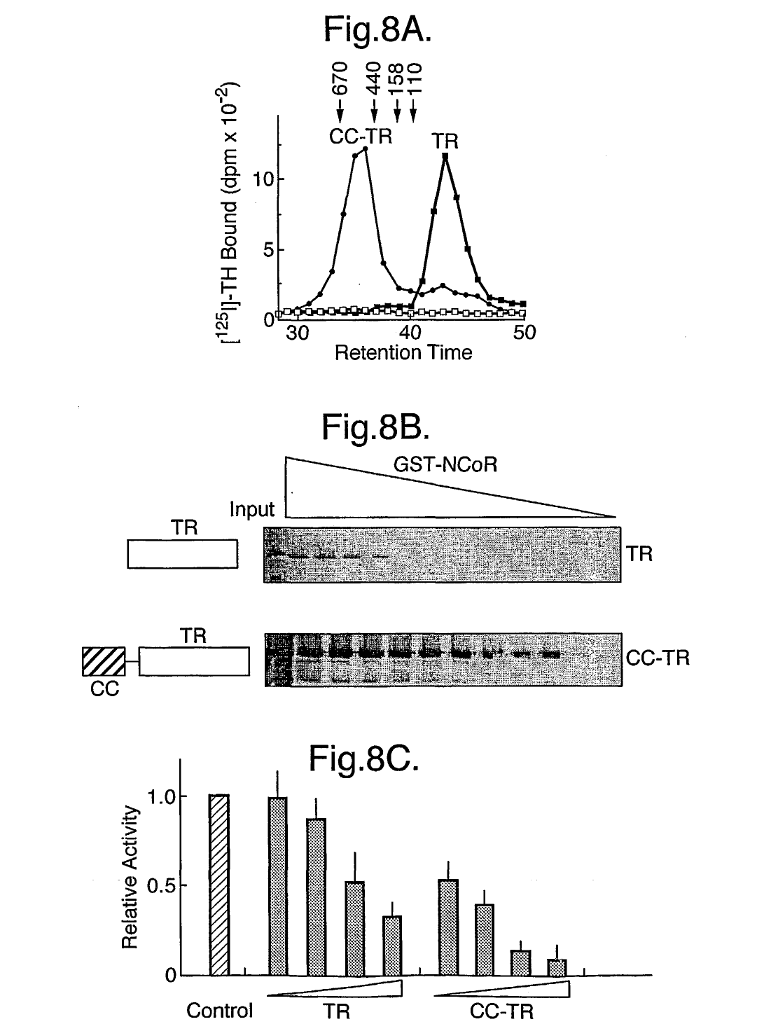

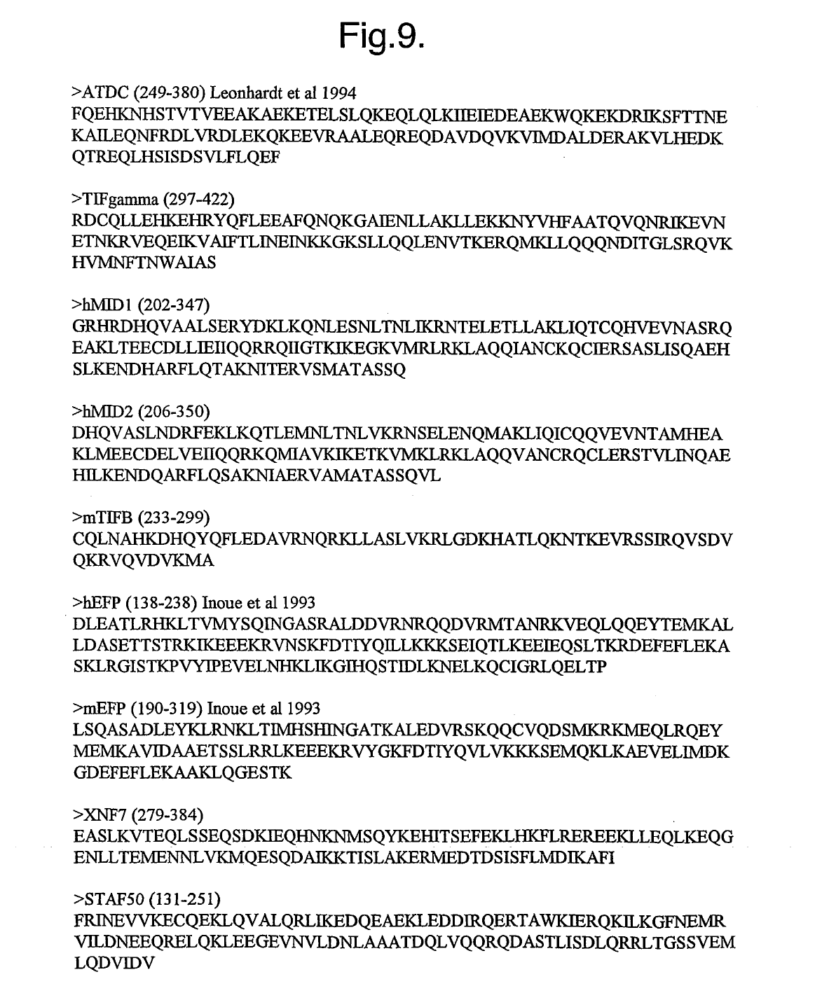

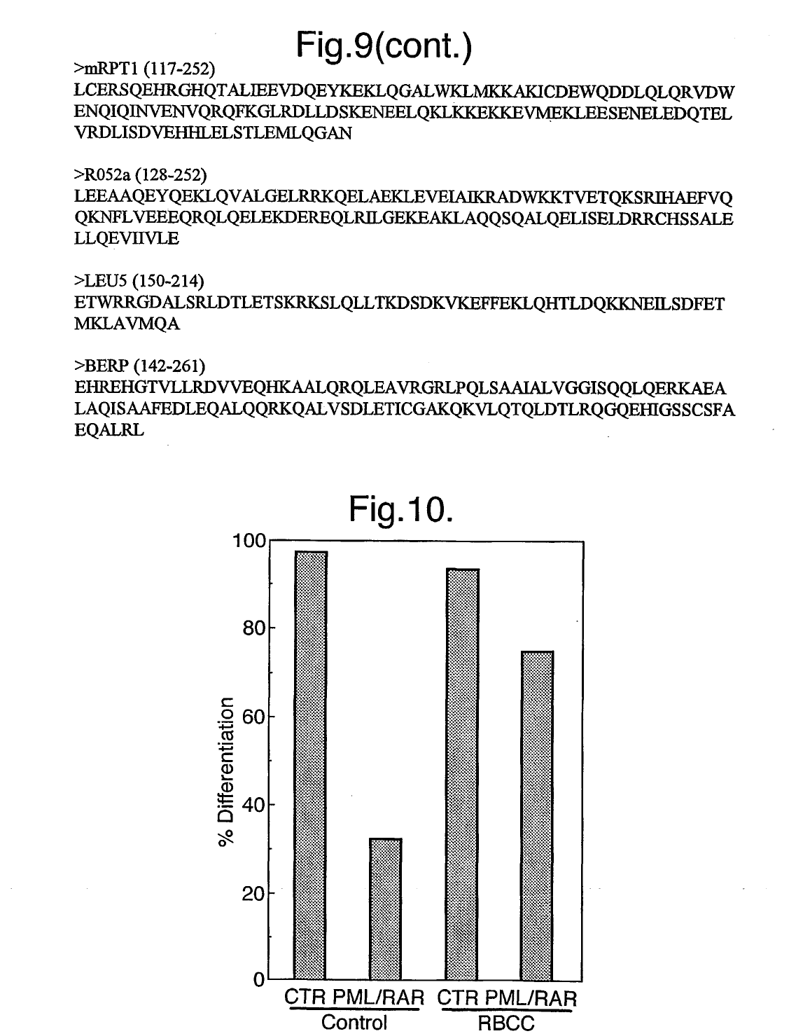

The inventors' findings point to a critical role for the fusion of the coiled coil domain of PML in altering the functional activity and inducing the oncogenic potential of RAR. To investigate if oligomerization would likewise enhance/alter the function of other factors, they generated a chimeric protein where the coiled coil domain of PML was fused to the entire coding sequence of the human thyroid receptor (TR). TR belongs to the superfamily of nuclear hormone receptors, and—as RAR—repress transcription by recruiting NCoR/HDAC in the absence of ligand. CC-TR was found in HMW complexes after gel filtration of in vitro translated reaction products, whereas TR eluted as a predominant monomeric fraction (FIG. 8A). This result shows that—upon fusion—the coiled coil region of PML is able to induce the formation of HMW complexes of heterologous factors. Interestingly, CC-TR showed a much stronger interaction with NCoR (FIG. 8B) and an enhanced capacity to repress transcription (FIG. 8C) compared to TR, indicating that oligomerization through the coiled coil enhanced its functional activity. Thus, oligomerization of a factor through fusion with the coiled coil of PML appears to be a (potentially) generally available method to enhance its function. Since coiled coil-medicated oligomers can form also in the extra-cellular environment (data not shown), this approach may be applicable to both intra- and extra-cellular peptides.

Disruption of PML-RAR Oligomers Prevents Differentiation Block

RAR oligomerization through the coiled coil region of PML is required for its leukemogenic activity, and deletion of the PML coiled coil is sufficient to lead to loss of oncogenic potential (Minucci et al., 2000). Small molecule compounds able to disrupt PML-RAR oligomers would therefore be able to revert the leukemic phenotype. As a proof of principle, we demonstrated here that over-expression of the coiled coil of PML (including the additional amino-terminal region required for targeting to the appropriate nuclear compartment)—by associating with PML-RAR and therefore reducing its oligomerization—achieved an anti-leukemic effect (FIG. 10).

The Capacity of Coiled Coil-Mediated Oligomers to Inactivate Cellular Proteins

Fusion of the PML coiled coil to a heterologous factor results in a chimeric protein with altered properties. In the case of PML-RAR and CC-RAR, the net outcome is a transcription factor with enhanced capacity to recruit co-regulators. So, it would appear that oligomerization has the capacity to enhance the biochemical properties of a given natural (or artificial) monomeric factor. Several factors (such as PML itself) are oligomeric in nature. In this case, addition of an extra-oligomerization interface would lead to formation (through an oligomerization chain reaction) of high-order oligomeric complexes, that may result in the formation of non-functional aggregates. The inventors investigated whether this may constitute a generally applicable approach for the functional inactivation of a given “target” protein, and termed this technology “RITA” (for “Reaching (protein) Inactivation Through Aggregation”). The oncosuppressor p53 protein forms tetramers, and oligomerization is required for its function. In the CC-p53 chimeric protein, the oligomerization domain of PML (CC) fused to the full-length coding sequence of p53 should impose an altered oligomerization state not only of the chimeric protein, but also of wild-type, interacting p53. In turn, this—according to the inventors' model—should lead to an improper organization of CC-p53/wt p53 hetero-oligomers, and to inhibition of p53 function.

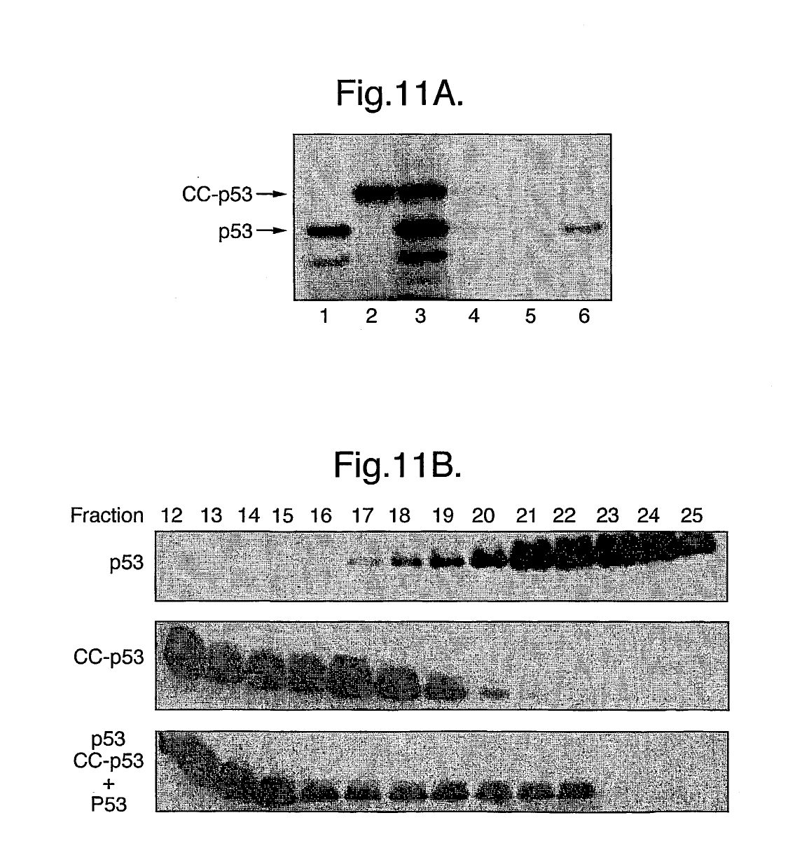

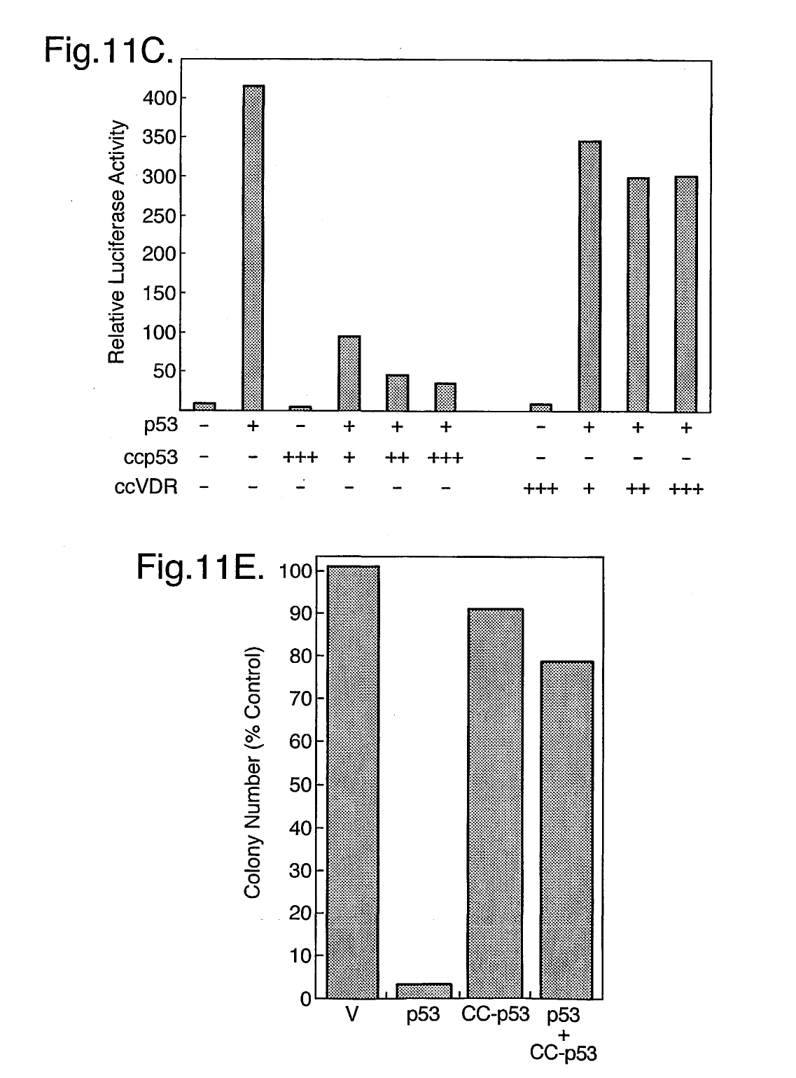

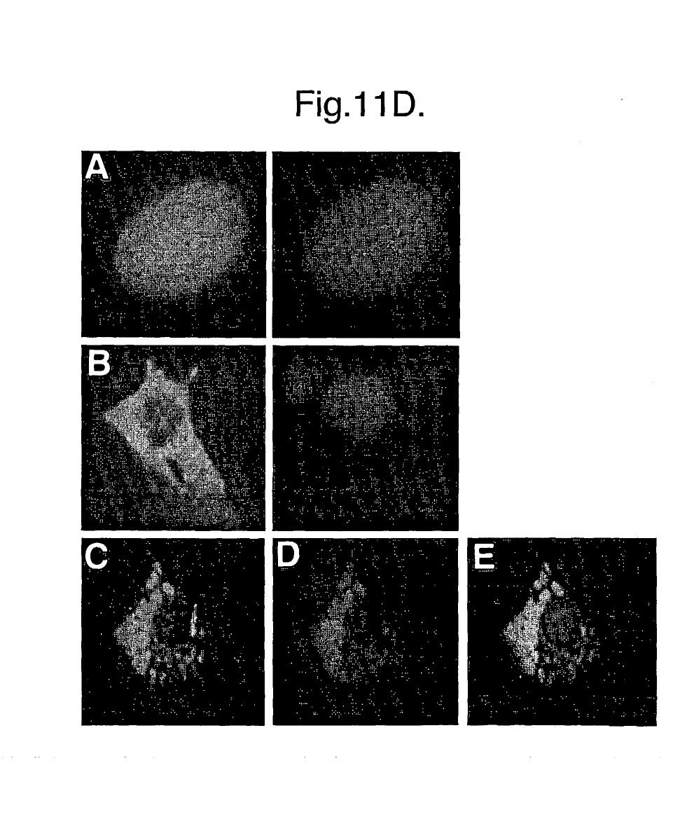

The inventors fused the CC region of PML to the full-length p53, to generate the chimeric CC-p53 protein. A necessary requirement for the chimeric CC-p53 protein would be the capacity to interact with wt p53, through the p53 tetramerization domain present in both proteins. Antibodies directed against the CC region of CC-p53 were able to immunoprecipitate in vitro translated p53 only in the presence of the CC-p53 chimera, showing the existence of a CC-p53/wt p53 complex (FIG. 11A).

P53 forms stable tetramers, as observed after size exclusion chromatography (SEC). Consistently with the presence of an additional oligomerization interface, CC-p53 is found in SEC fractions of much higher apparent molecular weight, of approximately 600 kDa (FIG. 11B). Given the capacity of CC-p53 to associate also with wt p53, the inventors measured the apparent molecular weight of the CC-p53/p53 hetero-oligomeric complex by SEC. Upon interaction with CC-p53, p53 was found to co-fractionate with the chimeric protein, (FIG. 11B). Co-immunoprecipitation experiments performed on the pooled fractions corresponding to the peaks of CC-p53 and wt p53 showed the formation of a hetero-oligomeric CC-p53/p53 complex, demonstrating that CC-p53 is able to recruit p53 into high-order oligomers (data not shown). The inventors did not notice a change in the elution profile of the CC-p53 chimera upon co-expression of the p53, suggesting that the hetero-oligomeric CC-p53/p53 complexes do not differ significantly from CC-p53 oligomers in size. To evaluate the transcriptional properties of CC-p53, the inventors performed transient transfection assays in murine embryonic fibroblasts (MEFs) derived from p53-/-mice. In these cells, transfection of a p53 reporter construct resulted in minimal levels of transcriptional activity (FIG. 11C). Co-transfection of an expression vector for wt p53 caused a strong increase in transcriptional activity of the reporter construct (50-100 fold: FIG. 11C). Co-transfection of an expression vector for CC-p53, in contrast, had no effect on reporter activity, showing that the chimeric protein is no longer able to regulate p53 target genes (FIG. 11C). Interestingly, co-transfection of increasing amounts of CC-p53 with wt p53 (at a fixed amount) resulted in a dramatic repression of wt p53 transcriptional activity (FIG. 11C). As a control, the chimeric CC-VDR protein, encoding for an unrelated transcription factor (vitamin D receptor) fused to the PML coiled coil, had little or no effect on p53 wt transcriptional activity (FIG. 11C). It appears therefore that CC-p53, interacting with wt p53, is able to block its transcriptional activity. Next, the investigated more in details the mechanism(s) underlying the dominant negative effect of CC-p53 over the wt p53 protein. The wt p53 protein is post-translationally regulated at several levels: stability, phosphorylation, acetylation.

The inventors first asked whether the hetero-oligomeric CC-p53/wt p53 complexes are less stable than the wt p53 protein: Western blot analysis of cells transiently transfected with the expression vector for wt p53, or co-transfected with the expression vectors for wt p53 and CC-p53, showed no significant difference in wt p53 levels, suggesting that CC-p53 is not targeting wt p53 for degradation(in conditions where p53 transcriptional activity is strongly repressed: data not shown). Next, The inventors checked for proper localization of the hetero-oligomeric complexes. NIH 3T3 cells were transiently transfected with expression vectors for wt p53, CC-p53, and—in some experiments—a GFP-p53 fusion protein, to allow visualization of p53 prior fixation of the cells, and to distinguish unambiguosly p53 from CC-p53. GFP-p53 behaves identically to wt p53 in all functional assays tested (data not shown). GFP-p53 and p53 displayed a typical, nuclear localization pattern (FIG. 11D, panel A, and data not shown). In contrast, CC-p53 was almost entirely localized in the cytoplasm (FIG. 11D, panel B). Strikingly, CC-p53 caused complete delocalization of either wt p53, or GFP-p53 (FIG. 11D, panels C-E). These results suggest that the dominant negative effect of CC-p53 over. wt p53 is mainly achieved through formation of hetero-oligomeric complexes unable to enter the nucleus.

Finally, the inventors measured the capacity of CC-p53 to inhibit the biological function of p53. Expression of wt p53 in p53 null SAOS cells results in cell growth arrest, apoptosis, and loss of colony forming capacity (FIG. 11E). CC-p53 had no effect on cell viability (FIG. 11E). Strikingly, CC-p53 almost completely abrogated the growth suppression effect by wt p53, demonstrating that the dominant negative effect on wt p53 activity is sufficient to inhibit its biological function (FIG. 11E).

Taken together, these results show that addition of the coiled coil domain of PML to a “target” protein results in functional inactivation of the target. The inventors have shown that in the case of proteins oligomeric in nature (such as wt p53), addition of an extra oligomerization domain (the coiled coil of PML) results in an oligomerization chain reaction not compatible with normal p53 localization and function. The RITA technology may therefore be applied to inactivate natural oligomeric proteins. Since the inventors have observed that the CC domain may mediate oligomerization also in the extracellular environment, this technique may be applied to both intra- and extra-cellular target proteins.

Discussion

The main results presented here are that PML-RAR forms nuclear oligomers in vivo, and that oligomerization of RAR (through fusion with the PML coiled coil region or with the p53 oligomerization domain) leads to deregulated transcription from RA-target promoters and differentiation block when expressed into primary hematopoietic progenitor cells. The present inventors propose that oligomerization is the mechanism responsible for the oncogenic activation of RAR upon fusion with PML.

As effectors of the RA signal, natural RARs directly regulate the expression of a variety of target genes, both in the absence (as repressors) and in the presence (as activators) of ligand (Minucci and Pelicci, 1999). Transcription from RA-target genes is an even more complex phenomenon, since several other intracellular signaling pathways and transcription factors contribute to their regulation (Mangelsdorf and Evans, 1995; Minucci and Ozato, 1996). Therefore, transcription from RA-target genes represents, at any given time-point and for each target promoter, the result of a “concerted” mode of transcriptional regulation, resulting from the cooperation among different DNA-binding proteins and associated co-regulators (Kadonga, 1998; Ptashne and Gann, 1997; Tjian and Maniatis, 1994). The inventors' findings show that oligomerization is sufficient to subvert this regulatory network by markedly enhancing the capacity of a transcription factor to recruit co-regulators, and leads to an “a solo” mode of deregulated transcription. The inventors hypothesize that oligomerization of RAR results in the recruitment of over-physiological concentrations of transcriptional corepressors, leading to a chromatin configuration which may render the target promoter's refractory to activating signals from other cis-regulatory elements (constitutive transcriptional repression). This model represents the explanation at the molecular level of the oncogenic activation of RAR in APL, and a framework for the future analysis of expression patterns of target genes deregulated by the fusion protein.

In several cases, transcription factors have been shown to oligomerize physiologically. Examples are p53, STAT5, Groucho, TEL, Sp1 (Clore et al, 1994; Chen et al, 1998; John et al, 1999 Jousset et al, 1997) and, as shown here, PML, PLZF and ETO. Regardless of their implications for leukemogenesis, the inventors' findings with PML-RAR and AML 1-ETO provide genetic evidence to demonstrate that oligomerization per se has profound effects on the regulatory properties of a transcription factor, to the point of radically modifying its biological effects. The L\PC-AML1-ETO deletion derivative (that cannot form HMW complexes) is still competent to bind NCoR, but is unable to repress transcription from an AML1 target gene (and to block differentiation). This finding demonstrates that the self-association domain of ETO is essential in directing efficient recruitment of the NCoR/HDAC complex and transcriptional repression. ETO is a transcription factor that physiologically forms HMW complexes and recruits the NCoR-HDAC complex. Although the natural targets of ETO are still unknown, the data presented here predicts that oligomerization is crucial for the natural function of ETO. Therefore, increased density of interacting domains for transcriptional coregulators, owing to formation of oligomeric complexes, may constitute a general mechanism to generate high local concentrations of coregulators. Notably, a single point mutation that prevents STAT5 tetramerization decreases levels of STAT5-mediated transcriptional activation (John et al., 1999).

A corollary derivable from these considerations: to function efficiently as “catalytic” centres for recruitment of coregulators, the interactions underlying the formation of the oligomeric complexes must be considerably tighter than those responsible for coregulator recruitment. The PML-RAR HMW complexes, but not the associations of RAR or PML-RAR with NCoR/HDAC, were resistant to strong lysis conditions, such as high salt (2M KCl), detergents (1% Triton or NP-40), reducing agents (50 mM DTT) (unpublished data), suggesting that coiled coil-mediated interactions are considerably more stable than the RAR-NCoR interactions. Consistently, in the experimental conditions used for gel filtration chromatography, the inventors did not find evidence of RAR-associated coregulators in HMW PML-RAR complexes (RAR itself was found to fractionate as a monomer). However, oligomerization and strength of self-association are not determined only by coiled coil structures. The p53 tetramerization domain, as compared to the PML coiled coil region, conferred similar biochemical, transcriptional and biological properties upon fusion with RAR. The APL fusion proteins PLZF-RAR and NPM-RAR formed HMW complexes. However, neither PLZF nor NPM have predicted coiled coil regions in their sequences. PLZF contains a BTB-POZ domain which, in the context of the GAGA transcription factor, mediates the formation of oligomeric complexes (Katsani et al., 1999); NPM contains an alternative amino-terminal oligomerization domain, that mediates the formation of hexameric structures (Chan and Chan, 1995). Not tested here, NuMA-RAR, another fusion protein of RAR found in one case of APL, also contains a strong oligomerization domain in the NuMA moiety of the fusion protein (Harborth et al., 1999).

However, the extreme heterogeneity of the protein-protein interaction modules that are apparently competent for oligomerization points to additional functions of these modules within the HMW complexes. The PML coiled coil- and the p53-RAR fusion proteins had identical transcriptional repression properties and effects on differentiation. However, only the coiled coil-RAR fusion was able to mount a RA response. The PML coiled coil region can also direct the formation of PML/PML-RAR hetero-oligomeric complexes and PML itself has been shown to function as a co-factor in the RA pathway (Wang et al., 1998) and to associate with histone acetylases (Doucas et al., 1999). Therefore, the RA-response that is observed in PML-RAR expressing cells might be a consequence of the unique ability of the PML coiled coil region to recruit wild-type PML proteins to PML-RAR oligomers.

The leukemia-associated fusion proteins always contain at least one transcription factor. The present inventors have shown for the first time that oligomerization, per se, is sufficient to activate the oncogenic potential of a transcription factor (RAR); that two leukemia-associated fusion proteins (PML-RAR and AML 1-ETO) exist in vivo as oligomeric complexes; and that in both these cases oligomerization is indispensable for oncogenesis. Oligomerization of transcription factors might, therefore, serve as a general mechanism of oncogene activation in leukaemias. TEL is a member of the Ets family of transcription factors, which contains an oligomerization domain and is found in the leukaemia-associated TEL-AML1 fusion protein. The TEL oligomerization domain is conserved in TEL-AML1 and is required for its transcriptional repressive properties (Jousset et at., 1997; Uchida et al., 1999). Notably, the portion of AML1 retained in this fusion includes a carboxy-terminal region lost in AML1-ETO and recently shown to recruit the Groucho family of co-repressors, suggesting that oligomerization might lead, also in this case, to constitutive transcriptional repressive activity of the fusion protein (Dittmer and Nordheim, 1998; Jousset et al., 1997; Levanon et al., 1998; Uchida et al., 1999). The oligomerization domain of TEL is also found in other leukemia-associated fusion proteins together with tyrosine-kinases (Platelet-derived growth factor receptor β or JAK2). In these cases, however, oligomerization leads to the constitutive activation of the associated tyrosine kinase (Carroll et al., 1996; Lacronique et al., 1997), a well-characterized and frequent mechanism of oncogene activation in human tumours. Therefore, oligomerization appears to be a mechanism of oncogene activation for both tyrosine kinases and transcription factors. Alterations of the oligomerization status of fusion proteins, containing either tyrosine kinases or transcription factors, are then expected to affect their oncogenic potential. Interestingly, oligomerization inhibitory peptides are able to revert in vitro the transforming phenotype of BCR/ABL, a tyrosine kinase fusion protein found in chronic myelogenous leukemia (Guo et al., 1998).

In conclusion, the present inventors have established for the first time the mechanism of altered recruitment of the NCoR/HDAC complex by PML-RAR in APL, and presented evidence suggesting that oligomerization of a transcription factor represents a potentially widespread mechanism of transcriptional regulation and oncogenic transformation. They have additionally shown that the approach of fusing a heterologous oligomerization domain (preferably, the coiled coil domain of PML) to a target protein may result paradoxically (given their observation that a similar phenomenon occurs in an oncogenic protein) in desirable properties for the said modified “target”, resulting in i) either a target with enhanced functional activity, or ii) in a target with impaired function, depending on the properties of the target prior modification. Thus, the inventors have established the theoretical and experimental basis for an approach that may have several applications in the biotechnology field, and in the design of novel therapies against various forms of diseases.

Materials and Methods

Plasmids

The following plasmids have been previously described: pSG5-PML-RAR, pSG5-PML-RAR AHT, pSG5-RAR, pSG5-RAR AHT, pSG5-ΔCC-PML-RAR, pSG5-CC-RAR, pGEX-NCoR (1782-2453), pCMV-GAL4-VP16, pcDNA3-PML, pcDNA3-NPM, pcDNA3-PLZF, pcDNA3-PLZF-RAR, pcDNA3-NPM-RAR. pcDNA3-AML1, pcDNA3-ETO, pcDNA3-myc-AML1-ETO(Gelmetti et al., 1998; Grignani et al., 1996; Lillie and Green, 1989; Zhang et al., 1997). pMAL-PML-RAR was obtained by site-directed mutagenesis of the 1^{st} ATG of the PML-RAR cDNA (from pSG5-PML-RAR) and insertion of an EcoRI site used for in-frame cloning in pMAL-C2 (New England BioLabs). pcDNA3-p53-RAR was cloned by insertion of a PCR fragment containing the tetramerization domain of p53 (Chen et al., 1998; Clore et al., 1994) carrying an optimal Kozak sequence and flanked by the appropriate restriction sites for in-frame cloning at the ATG of pSG5-RAR. pcDNA3-ΔPC-myc-AML1-ETO, pcDNA3-ΔZF-myc-AML-ETO were obtained by PCR-mediated deletion mutagenesis of the indicated regions of pcDNA3-AML1-ETO as described in the Results section (Gelmetti et al., 1998; Lutterbach et al., 1998; Lutterbach et al., 1998). PSG5-CC-p53 and pSG5-CC-RAR were obtained by replacing the RAR fragment from the pSG5-CC-RAR construct with a p53 fragment (Pearson et al., 2000), or a TR cDNA, both carrying a mutated ATG for in-frame cloning at the EcoRV site of pSG5-CC-RAR. The retroviral vectors were cloned by insertion of the appropriate cDNAs into the EcoRI site of Pinco (Grignani et al., 1998). pRARE-G5-TATA was obtained by inserting five GAL4 binding sites and the minimal promoter sequence from pG5E1b into the Xhol-Hindlll sites of the pGL2 plasmid-Promega- (Lillie and Green, 1989). Oligonucleotides containing the RARE from the RARβ2 promoter (Minucci et al., 1994) were inserted at the Mlu1 site. MDR1-luc was obtained by PCR of the MDR1 promoter region (Lutterbach et al., 1998) from a genomic clone and subsequent cloning in pGL2. All of the constructs have been verified by sequencing.

Pull-Down Assays and Co-Immunoprecipitation Experiments

GST-NCoR (1782-2453) or GST-NCoR (RDIII) purification and in vitro interaction experiments were performed as described, incubating the indicated amounts of GST-NCoR attached to a constant amount of glutathione-agarose beads in the presence of the appropriate ³⁵-S labelled, in vitro translated proteins(Gelmetti et al., 1998; Grignani et al., 1998; Zamir et al., 1996). The input lanes represent 100% of the total. Coimmunoprecipitation experiments were performed as described(Gelmetti et al., 1998), using extracts from transiently transfected COS-1 cells, or in vitro translated products.

Transactivation Assays

Transient transfection of HeLa, NIH 3T3, SAOS and C33A cells was performed by calcium phosphate as described (Lillie and Green, 1989; Minucci et al., 1994). p53-/- MEFs were transiently transfected by lipofection as described (Pearson et al., 2000). Light units were normalized to expression of a co-transfected β-galactosidase expression plasmid. Results are presented as the mean+standard deviations of at least three independent experiments.

Gel Filtration Analysis of RA Binding Activity.

Nuclear extracts (1-1.5 mg) from U937 cells or from transiently transfected COS-1 cells were incubated with 5 nM [³H]-RA (NEN Life Science) for 18 hrs at 4° C. [³H]-RA binding was analyzed using a gel filtration size exclusion column Superose 6 HR 10/30 (Pharmacia, Uppsala, Sweden) equilibrated in column buffer (Hepes 20 mM pH 7.4, EDTA 1 mM, DTT 1 mM, aprotinin and leupeptin 10 μg/ml, pepstatin 2 μg/ml, 1 mM PMSF, glycerol 1%, NaF 5 mM, KCl 0.4M). The [³H]-RA binding profile was measured using a Ramona 5 Radioactivity Monitoring radioflow detector analyser (Ray test, Milano, Italy) using a splitting device, electronically connected with the fraction collector.

Biochemical Purification of PML-RAR HMW Complexes and Size Exclusion Chromatography (SEC)

Nuclear extracts from U937 cells stably expressing PML-RAR (PR9 clone) were prepared as described (Nervi et al., 1992). Extracts were partially purified onto a Heparin-Sepharose column and then loaded on a Superose 6 HR 10/30 gel filtration column equilibrated in column buffer. The PML-RAR-containing fractions were diluted with incubation buffer lacking KCl (Hepes 20 mM pH 7.4, EDTA 1 mM, DTT 1 mM, aprotinin and leupeptin 10 μg/ml, pepstatin 2μg/ml, 1 mM PMSF, glycerol 10%, MgCl₂ 3 mM, NaF 5 mM, NP40 0.1%; final 0.1M KCl) and incubated for 1 hour at 4° C. with biotynilated RARE double-stranded oligonucleotide (7 μg/mg starting material) coupled to streptavidine-agarose beads as described (Blanco et al., 1998; Minucci et al., 1994). As controls, the inventors used either streptavidine-agarose beads alone, or performed the incubation with RARE-containing beads in the presence of a 100 fold excess RARE competitor in solution. Beads were eluted in buffer containing 1M KCl: aliquots of the eluted material (corresponding to approximately 10% of the PML-RAR amount present in the nuclear extracts) were analyzed by SDS-PAGE followed by silver stain or Western blotting, or were re-loaded onto a Superose 6 gel filtration column and then analyzed by Western blotting.

Expression and Characterization of Recombinant PML-RAR

pMAL-PML-RAR was expressed in BL21 cells. Bacterial lysates were incubated with amylose beads for two hours at 4° C. MBP-PML-RAR was eluted by adding maltose (20 mM), loaded onto a MonoQ column (SMART system, Pharmacia Biotech), and then subjected to an additional round of amylose affinity chromatography. MBP-PML-RAR was eluted and incubated with factor Xa to cleave the MBP moiety and yield purified PML-RAR, that was subsequently analysed by gel filtration chromatography (Superose 6 column, SMART system, Pharmacia Biotech).

In Vivo Cross/Inking Experiments

U937 PR9 cells were grown for 1 hour in medium devoid of cysteine and methionine, and then incubated for 8 hours in the presence of ³⁵-S labelled cysteine and methionine (Amersham). Before harvesting, cells were incubated for 30 minutes at room temperature in PBS plus 0.1 mM DTBP (Dimethyl 3,3′- dithiobisproprionamidate-2HCl, Pierce). Isolated nuclei were extracted in modified RIPA buffer (150 mM NaCl, 1% Nonidet P-40, 1% sodyum deoxycholate, 0.2% SDS, 2 mM EDTA, 5 mM NaF, aprotinin and leupeptin 10 μg/ml, pepstatin 2μg/ml, 1 mM PMSF, 100 mM Tris-Cl pH 7.4). The extracts were collected and analysed by gel filtration chromatography on a Superose 6 HR 10/30 column (Pharmacia Biotech) equilibrated and calibrated with globular molecular weight markers (Pharmacia Biotech) in the same buffer used for nuclear extraction. HMW PML-RAR complexes were immunoprecipitated with an anti-PML monoclonal antibody (Flenghi et al., 1995) or an unrelated antibody coupled to Protein G-Sepharose beads. The immunoprecipitated material was eluted from the beads in 1% SDS, and an aliquot was further analysed by gel filtration chromatography (Superose 6, SMART system, Pharmacia Biotech) to verify the integrity of the immunoprecipitated, cross-linked HMW complexes.

In Vitro Differentiation of Murine Hematopoietic Progenitor Cells Transduced with Retroviral Constructs

Murine hematopoietic progenitors were purified from the bone marrow of 12 weeks old BALB-C mice using commercially available kits (StemCell Tecnology). Cells were selected on the basis of the absence of lineage differentiation markers (lin−). Purified cells were pre-stimulated for two days in medium containing IL-3 (20 ng/ml), IL-6 (20 ng/ml) and stem cell factor (SCF, 100 ng/ml) and then attached to Retronectin (Takara Shuzo)-coated multiwell plates. Cells were incubated for 48 hours with the filtered supernatant from ecotropic packaging cells (Phoenix) transiently transfected with the indicated retroviral constructs (Grignani et al., 1998). Infected cells were sorted by FACS on the basis of their expression of GFP as a selectable marker from the vectors. Cells were seeded in methylcellulose plates (StemCell Tecnology) supplemented with IL-3, IL-6, and SCF as above and with the addition of G-CSF (60 ng/ml) and GM-CSF (20 ng/ml). After 8-10 days, cells were harvested and incubated with biotinylated anti Mac1 or GR1 antibodies (Pharmingen), followed by Cytochrome-C Streptavidin (Becton-Dickinson) and FACS analysis to evaluate the extent of differentiation.

Injection of Xenopus Oocytes and Mobility Shift Assays

Linearized plasmids were transcribed using a mMessage mMachine kit (Ambion) to produce capped mRNAs. Approximately 30 nl mRNA/Xenopus oocyte cytoplasm were injected as previously described (Minucci et al., 1998). The injected ooxytes were the incubated for 16 h at 18° C. and the protein expression evaluated by Western blot analysis. Mobility shift assays were performed in a mixture of 20 μl containing the specific DNA binding fragment, 0.5 μg of poly(dl:dC) in homogenization buffer as described (Landsberger and Wolffe, 1995; Minucci et al., 1998).

References

Ahmad, K. F ., et al. (1998). Proc Natl Acad Sci USA 95, 12123-8.

Benedetti, L., et al. (1997) Blood 90, 1175-85.

Blanco, J. C., et al (1998). Genes Dev 12, 1638-51.

Brown, D., et al. (1997). Proc Natl Acad Sci USA 94, 2551-6.

Carroll, M., et al. (1996). Proc Natl Acad Sci USA 93, 14845-50.

Chambon, P. (1996). Faseb J. 10,940-54.

Chakrabarti, S. R., and Nucifora, G. (1999). Biochem Biophys Res Commun 264, 871-7.

Chan, P. K., and Chan, F. V. (1995). Biochim Biophys Acta 1262, 37-42.

Chang, K. S., et al. (1995). Blood 85, 3646-53.

Chen, G., et al. (1998). Mol Cell Biol 18, 7259-68.

Cheng, G. X., et al. (1999). Proc Natl Acad Sci USA 96, 6318-23.

Clore, G. M., et al. (1994). Science 265, 386-91.

David, G., et al. (1998). Oncogene 16, 2549-56.

Dittmer, J., and Nordheim, A. (1998). Biochim Biophys Acta 1377, F1-11.

Doucas, V., et al. (1999). Proc Natl Acad Sci USA 96, 2627 -32.

Du, C., et al. (1999). Blood 94, 793-802.

Flenghi, L., et al. (1995) Blood 85, 1871-80.

Gelmetti, V., (1998). Mol Cell Biol 18, 7185-91.

Grignani, F ., et al. (1999). Oncogene 18, 6313-21.

Grignani, F., et al. (1998). Nature 391, 815-8.

Grignani, F., et al. (1993). Cell 74, 42331.

Grignani, F., et al. (1998). Cancer Res 58, 14-9.

Grignani, F., et al. (1996). Embo J 15, 4949-58.

Grisolano, J. L., et al. (1997). Blood 89, 376-87.

Grunstein, M. (1997). Nature 389, 349-352.

Guidez, F., et al. (1998). Blood 91, 2634-42.

Guo, X. V., et al. (1998). Oncogene 17, 825-33.

Harborth, J., et al. (1999). Embo J 18, 1689-700.

He, L. Z., (1998). Nat Genet 18, 126-35.

Hirano, T., and Mitchison, T. J. (1994). Cell 79,449-58.

Horlein, A. J., et al. (1995). Nature 377, 397-404.

Inoue et al (1993) Proc. Natl. Acad. Sci. U.S.A. 90, 11117-11121.

John, S., et al. (1999). Mol Cell Biol 19, 1910-8.

Jousset, C., et al. (1997). Embo J 16, 69-82.

Kadonaga, J. T. (1998). Cell 92, 301-13.

Kammerer, R. A., et al. (1998). J Biol Chem 273, 10602-8.

Katsani, K. R., et al. (1999). Embo J 18, 698-708.

Kitabayashi, I., et al. (1998). Embo J 17, 2994-3004.

Lacronique, V., et al. (1997). Science 278, 1309-12.

Landsberger, N., and Wolffe, A. P. (1995). Semin Cell Biol 6, 191-9.

Lavau, C., et al. (1997). Embo J 16, 4226-37.

Leonhardt et al., Genomics (1994) 19, 130-136.

Levanon, D., et al. (1998). Proc Natl Acad Sci USA 95, 11590-5.

Lillie, J. W., and Green, M. R. (1989). Nature 338, 39-44.

Lin, R. J., et al. (1998). Nature 391, 811-4.

Look, A. T. (1997). Science 278, 1059-64.

Lupas, A. (1996). Trends Biochem Sci 21, 375-82.

Lutterbach, B., et al. (1998). Mol Cell Biol 18, 3604-11.

Lutterbach, B., et al. (1998). Mol Cell Biol 18, 7176-84.

Mangelsdorf, D. J., and Evans, R. M. (1995). Cell 83, 841-50.

McWhirter, J. R., et al. (1993). Mol Cell Biol 13, 7587 -95.

Melnick, A., and Lrcht, J. D. (1999). Blood 93, 3167-215.

Meyers, S., et al. (1995). Mol Cell Biol 15, 1974-82.

Minucci, S., et al. (1994). Mol Cell Biol. 14, 360-72.

Minucci, S., and Ozato, K. (1996). Curr Opin Genet Dev 6, 567-74.

Minucci, S., et al. (1998). Mol Endocrinol. 12, 315-24.

Minucci, S., and Pelicci, P. G. (1999). Semin Cell Dev. Biol. 10, 215-25.

Nervi, C., et al. (1992). Cancer Res 52, 3687-92.

Okuda, K., et al. (1997). J Clin Invest 100, 1708-15.

Pazin, M., and Kadonaga, J. T. (1997). Cell 89, 325-328.

Pearson, M et al. (2000). Nature, 406, 207-210.

Pereira, D. S., et al. (1998). Proc Natl Acad Sci USA 95, 8239-44.

Ptashne, M., and Gann, A. (1997). Nature 386, 569- 77.

Purton, L. E., et al. (1999). Blood 94, 483-95.

Rabbitts, T. H. (1994). Nature 372, 143-9.

Rabbitts, T. H. (1991). Cell 67, 641-4.

Redner, R. L., et al. (1999). Blood 94, 417 -28.

Ruthardt, M., et al. (1997). Mol Cell Biol 17, 4859-69.

Schwaller, J., et al. (1998). Embo J 17, 5321-33.

Shivdasani, R. A., and Orkin, S. H. (1996). Blood 87, 4025-39.

Siegel, L. M., and Monty, K. J. (1966). Biochim Biophys Acta 112, 346-62.

Slany, R. K., et al. (1998). Mol Cell Biol. 18, 122-9.

Stunnenberg, H. G., et al. (1999). Biochim Biophys Acta 1423, F15-33.

Saurin et al Trends Biochem Sci, 21, 208-214 (1996)

Tenen, D. G., et al. (1997). Blood 90, 489-519.

Tjian, R., and Maniatis, T. (1994). Cell 77, 5-8.

Uchida, H., et al. (1999). Oncogene 18, 1015-22.

Wang, J., et al. (1998). Proc Natl Acad Sci USA 95, 10860-5.

Wang, Z. G., et al. (1998). Science 279, 1547-51.

Westervelt, P., and Ley, T. J. (1999). Blood 93, 2143-8.

Wolffe, A. P., et al. (1997). Biochem Soc Trans 25, 612-5.

Xu, L., et al. (1999). Curr Opin Genet Dev 9, 140-7.

Zamir, I., et al. (1996). Mol Cell Biol. 16, 5458-65.

Zhang, V. W., et al. (1997). Mol Cell Biol. 17, 4133-45.

1. A method of screening for a substance having the ability to modulate the oligomerization domain of an oligomeric factor such that strong self-association of the oligomeric factors to form oligomeric complexes is prevented or reduced, said method comprising the steps of
(a) bringing into contact a first oligomeric factor or the functional self-association part thereof, a second oligomeric factor or the functional self association part thereof, and a test substance, under conditions wherein, in the absence of said test substance, being an inhibitor of association of said oligomeric factors, said oligomeric factors or functional self associating parts thereof interact or bind; and
(b) determining the interaction or binding between said oligomeric factors or functional self association parts thereof.

2. A method of screening for a test compound able to bind an oligomerization domain of an oligomeric factor, said method comprising the steps of
(a) bringing into contact a substance which includes an oligomerization domain which allows self-association of the oligomeric factors, or a variant, derivative or analogue thereof, and a test compound, and;
(b) determining binding between said oligomerization domain and the test compound.

3. A method according to claim 1 or claim 2 wherein the oligomeric factor is a fusion protein comprising at least one transcription factor.

4. A method according to claim 3 wherein the oligomeric factor is PML-RAR or AML1-ETO.

5. A method according to claim 1 further comprising the steps of isolating said test substance and manufacturing a medicament comprising the isolated test substance for use in treating a disease associated with the formation of HMW complexes of oligomeric factors.

6. A method according to claim 2 further comprising the steps of isolating said test compound and manufacturing a medicament comprising the isolated test compound for use in treating a disease associated with the formation of HMW complexes of oligomeric factors.

7. A method according to claim 5 or claim 6 wherein the disease is cancer.

8. A method according to claim 6 wherein the test compound is an antibody binding domain.

9. A method of increasing the activity of a monomeric polypeptide in a sample, comprising the steps of producing a chimeric protein comprising the polypeptide and an oligomerization domain, and adding said chimeric protein to the sample comprising monomeric polypeptides thereby allowing self-association of the monomeric polypeptides to the chimeric protein and increasing the activity of the polypeptide in the sample.

10. A method according to claim 9 wherein the chimeric protein is a fusion protein comprising said polypeptide and an oligomerization domain.

11. A method according to claim 9 or claim 10 wherein the oligomerization domain is the coiled coil domain of PML.

12. A method according to claim 9 or claim 10 wherein the oligomerization domain is derived from p53, PLZF, NPM or ETO.

13. A method according to any one of claims 9 to 12 wherein the population of monomeric polypeptides in intracellular.

14. A method of reducing the activity of an oligomeric polypeptide, comprising the steps of producing a modified oligomeric polypeptide comprising said polypeptide and an additional oligomerization domain, and contacting said modified oligomeric polypeptide with a population of oligomeric polypeptides in a sample thereby allowing association of the oligomeric polypeptides to the modified oligomeric polypeptide and as a result decreasing the activity of the oligomeric polypeptide in the sample.

15. A method according to claim 14 wherein the modified oligomeric polypeptide is a fusion protein comprising said oligomeric polypeptide and an oligomerization domain.

16. A method according to claim 14 or claim 15 wherein the oligomerization domain is the coiled coil domain of PML.

17. A method according to any one of claims 14 to 16 wherein the oligomeric polypeptide is p53, cytokines, interleukins, or TNF.

18. Use of a factor capable of disrupting the activity or formation of HMW complexes in the preparation of a medicament for treating a disease associated with the formation of HMW complexes comprising oligomeric factors.

19. Use according to claim 18 wherein the oligomeric factors are chimeric transcription factors.

20. Use according to claim 19 wherein the factor is a binding member capable of specifically binding to the oligomerization domain of the chimeric transcription factor.

21. Use according to claim 19 or claim 20 wherein the oligomerization domain is a coiled coil domain.

22. Use according to any one of claims 19 to 21 wherein the disease is cancer, particularly leukaemia.

23. Use according to any one of claim 19 to 22 wherein the chimeric transcription factor is PML-RAR or AML1-ETO.

24. Use according to any of claims 19 to 23 wherein the binding member is a peptide comprising a coiled coil domain of the chimeric transcription factor.

25. Use according to claim 24 wherein the coiled coil domain has an amino acid sequence having at least 70% homology with the sequence identified in SEQ ID No. 1.

26. Use according to claim 25 wherein the coiled coil domain has an amino acid sequence having the sequence as shown in SEQ ID No. 1.

27. A method of determining the presence or absence of a HMW complex comprising two or more oligomeric factors, said method comprising the steps of obtaining a biological sample from a patient and detecting the presence or absence of said HMW complex using a specific binding member capable of specifically binding to said HMW complex.

28. A method according to claim 27 further comprising the step of determining the molecular weight of HMW complex detected in the biological sample.

29. A method according to claim 27 or claim 28 wherein the HMW comprises chimeric transcription factors.

30. A method according to claim 29 wherein the chimeric transcription factors. comprise PML-RAR or AML1-ETO.

31. A method treating a patient having, or suspected of having, a disease associated with the formation of HMW complexes comprising two or more factors capable of forming self-associating oligomers, said method comprising the steps of administering to said patient a substance capable of preventing and/or disrupting the activity or formation of said HMW complexes.

32. A method according to claim 31 wherein said substance is a binding member capable of specifically binding to the oligomerization domain of the oligomeric factor.

33. A method according to claim 31 or claim 32 wherein the oligomeric factors are chimeric transcription factors.

34. A method according to claim 33 wherein the chimeric transcription factor is PML-RAR or AML1-ETO.

35. A method according to any one of claims 31 to 34 wherein the disease is cancer.

36. A method according to claim 35 wherein the disease is leukaemia.

37. A compound for use in modulating the activity of a polypeptide, said compound comprising said polypeptide fused to an oligomerization domain of an oligomeric protein.

38. A compound according to claim 37 wherein the oligomerization domain is the coiled coil domain and the oligomeric protein is PML.

39. A compound according to claim 37 or claim 38 wherein the polypeptide is a monomeric polypeptide and the activity of said polypeptide is increased.

40. A compound according to claim 37 or claim 38 wherein the polypeptide is oligomeric in nature and the activity of the polypeptide is reduced.

41. A pharmaceutical composition comprising a compound according to any one of claims 37 to 40 and a pharmaceutically acceptable recipient.
